SEQUENCE LISTING (30 sequences)

SEQ ID NO: 1 - protein, 23 aa, Streptococcus pyogenes
VDSPIEQPRIIPNGGTLTNLLGN

SEQ ID NO: 2 - protein, 58 aa, Streptococcus pyogenes
VDSPIEQPRIIPNGGTLTNLLGNAPEKLALRNEERAIDELKKQAIEDKEATTAIEAAS

SEQ ID NO: 3 - protein, 28 aa, Streptococcus pyogenes
SDALEALADQTDALQSEEAAVVKADNAA

SEQ ID NO: 4 - protein, 184 aa, Streptococcus pyogenes
VDSPIEQPRIIPNGGTLTNLLGNAPEKLALRNEERAIDELKKQAIEDKEATTAIEAASSDALEALADQTDALQSEEAAVVKADNAASDALEALADQTDALQSEEAEVVQSDNAASDAWEKAATPIALDVKKTKDTKPVVKKEERQNVNTLPTTGEESNPFFTAAALAIMVSTGVLVVSSKCKEN

SEQ ID NO: 5 - protein, 141 aa, Streptococcus pyogenes
VDSPIEQPRIIPNGGTLTNLLGNAPEKLALRNEERAIDELKKQAIEDKEATTAIEAASSDALEALADQTDALQSEEAAVVKADNAASDALEALADQTDALQSEEAEVVQSDNAASDAWEKAATPIALDVKKTKDTKPVVKK

SEQ ID NO: 6 - protein, 159 aa, Streptococcus pyogenes
VDSPIEQPRIIPNGGTLTNLLGNAPEKLALRNEERAIDELKKQAIEDKEATTAIEAASSDALEALADQTDALQSEEAAVVKADNAASDALEALADQTDALQSEEAEVVQSDNAASDAWEKAATPIALDVKKTKDTKPVVKKEERQNVNTLPTTGEESNP

SEQ ID NO: 7 - protein, 217 aa, Streptococcus pyogenes
MGKEIKVKCFLRRSAFGLVAVSASVLVGSTVSAVDSPIEQPRIIPNGGTLTNLLGNAPEKLALRNEERAIDELKKQAIEDKEATTAIEAASSDALEALADQTDALQSEEAAVVKADNAASDALEALADQTDALQSEEAEVVQSDNAASDAWEKAATPIALDVKKTKDTKPVVKKEERQNVNTLPTTGEESNPFFTAAALAIMVSTGVLVVSSKCKEN

SEQ ID NO: 8 - protein, 259 aa, Streptococcus pyogenes
SAFGLVAVSASVLVGSTVSAVDSPIEQPRIIPNGGTLTNLLGNAPEKLALRNEERAIDELKKQAIEDKEATTAIEAASSDALEALADQADALQSEEAAVVQSDNAASDALEALADQTDALQSEEAAVVKADNAASDTLEALADQTDALQSEEAAVVKADNAASDTLEALADQTDALQSEEAAVVKADNAASDTLEALADQTDALQSEEAEVVQSDNAASDAWGKAATPIALDVKKTKDTKPVVKKEERQNVNTLPTTGE

SEQ ID NO: 9 - protein, 155 aa, Streptococcus pyogenes
DSPIEQPRIIPNGGTLINLLGNAPEKLALRNEERAIDELKKQAIEDKEATTAIEAASSDALEALADQTDALQSEEAAVVKADNAASDALEALADQTDALQSEEAEVVQSDNAASDAWEKAATPIALDVKKTKDTKPVVKKEERQNVNTLPTTGEE

SEQ ID NO: 10 - protein, 271 aa, Streptococcus pyogenes
VSAVDSPIEQPRIIPNGGTLTNLLGNAPEKLALRNEERAIDELKKQAIEDKEATTAIEAASSDALEALADQADALQSEEAAVVQSDNAASDALEALADQADALQSEEAAVVQSDNAAGDALEALADQTDALQSEEASVVKADNAASDALEALADQTDALQSEEASVVKADNAASDALEALADQTDALQSEEAAVVKADNAASDALEALADQTDALQSEEAEVVQSDNAASDAWEKAATPIALDVKKTKDTKPVVKKEERQNVNTLPTTGEE

SEQ ID NO: 11 - protein, 167 aa, Streptococcus pyogenes
ASVLVGSTVSAVDSPIEQPRIIPNGGTLTNLLGNAPEKLALRNEERAIDELKKQAIEDKEATTAIEAASSDALEALADQTDALQSEEAAVVKADNAASDALEALADQTDALQSEEAEVVQSDNAASDAWEKAATPIALDVKKTKDTKPVVKKEERQNVNTLPTTGEE

SEQ ID NO: 12 - DNA, 654 bp, Streptococcus pyogenes
atgggaaaagaaataaaagtgaaatgctttttgcgtagatcagcttttggattagttgcggtgtcagcatcagtattagtcggttcaacagtatctgctgttgactcacctatcgaacagcctcgaattattccaaatggcggaaccttaactaatcttcttggcaatgctccagaaaaactggcattacgtaatgaagaaagagccattgatgaattaaaaaaacaagctattgaggataaagaagctacgacagctatagaagcagcaagttcagatgccttagaagcattagcggatcaaacagacgctttacaatcagaagaagctgcggttgttaaagcggataacgctgctagtgacgccttagaagcattggcggatcaaacagacgctttacaatcagaagaagctgaagtagttcaatcagataacgctgctagtgacgcctgggaaaaagcagcaactccaatcgctttagatgttaagaaaactaaagatacaaaacctgtagttaaaaaagaagaaagacaaaacgttaatacccttcctacaactggtgaagagtctaacccattctttacagctgctgcgcttgcaataatggtaagtacaggtgtgttagttgtaagttcaaagtgcaaagaaaattag

SEQ ID NO: 13 - DNA, 777 bp, Streptococcus pyogenes
tcagcttttggattagttgcggtgtcagcatcagtattagtcggttcaacagtatctgctgttgactcacctatcgaacagcctcgaattattccaaatggcggaaccttaactaatcttcttggcaatgctccagaaaaactggcattacgtaatgaagaaagggccattgatgaattaaaaaaacaagctattgaggataaagaagctacgacagctatagaagcagcaagttcagatgccttagaagcattagcggatcaagcagacgctttacaatcagaagaagctgcagtagttcaatcagataacgctgctagtgacgccttagaagcattggcggatcaaacagacgctttacaatcagaagaagctgcggttgttaaagcggataacgctgctagtgacactttagaagcattggcggatcaaacagacgctttacaatcagaagaagctgcggttgttaaagcggataacgctgctagtgacactttagaagcattggcggatcaaacagacgctttacaatcagaagaagctgcggttgttaaagcggataacgctgctagtgacactttagaagcattggcggatcaaacagacgctttacaatcagaagaagctgaagtagttcaatcagataacgctgctagtgacgcctggggaaaagcagcaactccaatcgctttagatgttaagaaaactaaagatacaaaacctgtagttaaaaaagaagaaagacaaaacgttaatacccttcctacaactggtgaa

SEQ ID NO: 14 - DNA, 469 bp, Streptococcus pyogenes
gactcacctatcgaacagcctagaattattccaaatggcggaaccttaattaatcttcttggcaatgctccagaaaaactggcattacgtaatgaagaaagagccattgatgaattaaaaaaacaagctattgaggataaggaagctacgacagctatagaagcagcaagttcagatgccttagaagcattagcggatcaaacagacgctttacaatcagaagaagctgcggttgttaaagcggataacgctgctagtgacgccttagaagcattggcggatcaaacagacgctttacaatcagaagaagctgaagtagttcaatcagataacgctgctagtgacgcctgggaaaaagcagcaactccaatcgctttagatgttaagaaaactaaagatacaaaacctgtagttaaaaaagaagaaagacaaaacgttaatacccttcctacaactggtgaagagtaac

SEQ ID NO: 15 - DNA, 853 bp, Streptococcus pyogenes
gttgcggtgtcagcatcagtattagtcggttcaacagtatctgctgttgactcacctatcgaacagcctcgaattattccaaatggcggaaccttaactaatcttcttggcaatgctccagaaaaactggcattacgtaatgaagaaagagccattgatgaattaaaaaaacaagctattgaggataaagaagctacgacagctatagaagcagcaagttcagatgccttagaagcattagcggatcaagcagacgctttacaatcagaagaagctgcagtagttcaatcagataacgctgctagtgacgccttagaagcattagcggatcaagcagacgctttacaatcagaagaagctgcagtagttcaatcagataacgctgctggtgacgccttagaagcattggcggatcaaacagacgctttacaatcagaagaagcttcggttgttaaagcggataacgctgctagtgacgccttagaagcattggcggatcaaacagacgctttacaatcagaagaagcttcggttgttaaagcggataacgctgctagtgacgccttagaagcattggcggatcaaacagacgctttacaatcagaagaagctgcggttgttaaagcggataacgctgctagtgacgccttagaagcattggcggatcaaacagacgctttacaatcagaagaagctgaagtagttcaatcagataacgctgctagtgacgcctgggaaaaagcagcaactccaatcgctttagatgttaagaaaactaaagatacaaaacctgtagttaaaaaagaagaaagacaaaacgttaatacccttcctacaactggtgaagagtaac

SEQ ID NO: 16 - DNA, 504 bp, Streptococcus pyogenes
gcatcagtattagtgggttcaacagtatctgctgtggactcacctatcgaacagcctcgaattattccaaatggcggaaccttaactaatcttcttggcaatgctccagaaaaactggcattacgtaatgaagaaagagccattgatgaattaaaaaaacaagctattgaggataaagaagctacgacagctatagaagcagcaagttcagatgccttagaagcattagcggatcaaacagacgctttacaatcagaagaagctgcggttgttaaagcggataacgctgctagtgacgccttagaagcattggcggatcaaacagacgctttacaatcagaagaagctgaagtagttcaatcagataacgctgctagtgacgcctgggaaaaagcagcaactccaatcgctttagatgttaagaaaactaaagatacaaaacctgtagttaaaaaagaagaaagacaaaacgttaatacccttcctacaactggtgaagagtaa

SEQ ID NO: 17 - DNA, 24 bp, Artificial Sequence
  Description of Artificial Sequence  primer
agcttttggattagttgcggtgtc

SEQ ID NO: 18 - DNA, 27 bp, Artificial Sequence
  Description of Artificial Sequence primer
agcttttggattagttgcggtgtcagc

SEQ ID NO: 19 - DNA, 25 bp, Artificial Sequence
  Description of Artificial Sequence primer
ttgactcacctatcgaacagcctcg

SEQ ID NO: 20 - DNA, 32 bp, Artificial Sequence
  Description of Artificial Sequence primer
aaaacctgtagttaaaaaagaagaaagacaaa

SEQ ID NO: 21 - DNA, 22 bp, Artificial Sequence
  Description of Artificial Sequence primer
ccttcctacaactggtgaagag

SEQ ID NO: 22 - protein, 19 aa, Streptococcus pyogenes
DSPIEQPRIIPNGGTLTNC

SEQ ID NO: 23 - protein, 25 aa, Streptococcus pyogenes
EKLALRNEERAIDELKKQAIEDKEC

SEQ ID NO: 24 - protein, 19 aa, Streptococcus pyogenes
EKLALRNEERAIDELKKQC

SEQ ID NO: 25 - protein, 18 aa, Streptococcus pyogenes
EERAIDELKKQAIEDKEC

SEQ ID NO: 26 - protein, 20 aa, Streptococcus pyogenes
KKTKDTKPVVKKEERQNVNC

SEQ ID NO: 27 - DNA, 764 bp, Streptococcus pyogenes
tttccaaattatcggtaatttaatatgctaatgcatataaaaataaaaaaggagaaacaatgggaaaagaaataaaagtgaaatgctttttgcgtagatcagcttttggattagttgcggtgtcagcatcagtattagtcggttcaacagtatctgctgttgactcacctatcgaacagcctcgaattattccaaatggcggaaccttaactaatcttcttggcaatgctccagaaaaactggcattacgtaatgaagaaagagccattgatgaattaaaaaaacaagctattgaggataaagaagctacgacagctatagaagcagcaagttcagatgccttagaagcattagcggatcaaacagacgctttacaatcagaagaagctgcggttgttaaagcggataacgctgctagtgacgccttagaagcattggcggatcaaacagacgctttacaatcagaagaagctgaagtagttcaatcagataacgctgctagtgacgcctgggaaaaagcagcaactccaatcgctttagatgttaagaaaactaaagatacaaaacctgtagttaaaaaagaagaaagacaaaacgttaatacccttcctacaactggtgaagagtctaacccattctttacagctgctgcgcttgcaataatggtaagtacaggtgtgttagttgtaagttcaaagtgcaaagaaaattagttgctatttgttctagcaaatgaaacaagggaatcgaaagattctcttttt

SEQ ID NO: 28 - DNA, 23 bp, Artificial Sequence
  PCR primer
gactcacctatcgaacagcctcg

SEQ ID NO: 29 - DNA, 22 bp, Artificial Sequence
  PCR primer
agcttcttctgattgtaaagcg

SEQ ID NO: 30 - protein, 20 aa, Streptococcus pyogenes
QKQQQLETEKQISEASRKSC

(19) United States
(12) Patent Application Publication
(10) Pub. No.: US 2002/0061306 A1
(43) Pub. Date: May 23, 2002

(54) Streptococcal alpha ZM binding protein

(76) Inventors: Lars Bjorck, Lund (SE); Magnus Rassmussen, Lund (SE)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/847,539

(22) Filed: May 1, 2001

Related U.S. Application Data
(63) Continuation of application No. PCT/GB99/03631, filed on Nov. 2, 1999.

(30) Foreign Application Priority Data
Nov. 2, 1998 (GB) 9823975.9

(51) Int. Cl. A61K 39/40; C07K 16/12; C07H 21/04; C12P 21/02

(52) U.S. Cl. 424/150.1; 530/388.4; 536/23.53; 435/69.1

(57) ABSTRACT

A protein is described which is capable of binding to α₂ macro globulin. The protein comprises the amino acid sequence of SEQ ID No: 1 or a functional variant thereof. The invention also relates to a peptide comprising a fragment of the protein of at least six amino acids in length. A protein or peptide which is capable of generating a protective immune response to Group A streptococcus comprises the amino acid sequence of SEQ ID No: 1, a functional variant thereof or a functional variant of at least six amino acids in length of either thereof. Such a protein or peptide may be used in a vaccine composition together with a pharmaceutically acceptable carrier.

### FIELD OF THE INVENTION

The invention relates to a new family of proteins which are able to bind to α₂-macroglobulin and peptide fragments of this family of proteins. The invention also relates to the use of a protein or peptide derived from an α₂-macroglobulin binding protein for use in a vaccine composition for group A streptococcus.

### BACKGROUND OF THE INVENTION

Streptococcus pyogenes (group A Streptococcus) (GAS) is an important human pathogen which causes a variety of diseases such as pharyngitis, impetigo, scarlatina and erysipelas. More severe infections caused by this organism are necrotizing fasciitis and streptococcal toxic shock like syndrome.

S. pyogenes binds several human plasma proteins via its surface proteins. S. pyogenes binds to α₂ macroglobulin (α₂M) which is a proteinase inhibitor. α₂M is a glycoprotein of 718 kD composed of two pairs of identical subunits held together by disulphide bonds.

Previous studies have identified a non-proteolytic cell wall protein of 78 kD of Group A Streptococci which binds to α₂M: Chhatwal et al J. Bacteriol. (1987) 169(8) 3691-5.

### SUMMARY OF THE INVENTION

The present inventors have identified a new group of proteins which are expressed on the surface of some strains of Group A streptococcus, S. pyogenes. These proteins have the ability to bind to α₂-macroglobulin, and show some homology to protein G of Group G streptococcus. The new protein derived from S. pyogenes has been termed protein GRAB by the present inventors. The gene encoding this protein is referred to as grab.

The present invention relates in particular to a protein which is capable of binding α₂M and which comprises the amino acid sequence of SEQ ID No. 1 or a functional variant thereof. In preferred embodiments, the protein comprises the amino acid sequence of SEQ ID No. 2 or a functional variant thereof, and/or one or more tandem repeats having the amino acid sequence of SEQ ID No 3 or a variant thereof. The protein of the invention may further comprise a cell membrane anchor region and a hydrophobic transmembrane region. Preferably, the protein consists of the amino acid sequence of any of SEQ ID Nos. 1 to 11 and variants thereof.

The invention also provides:

a peptide comprising a fragment of at least 6 amino acids in Length of a protein having the amino acid sequence of (a) any of SEQ ID Nos 1 to 11 or (b) a variant of any of SEQ ID Nos 1 to 11;

a peptide as defined above having the ability to generate an immune response in an individual and vaccine compositions comprising such a peptide and methods of immunization comprising administering such a peptide to an individuals;

a DNA sequence which codes for a protein or peptide according to the invention, said DNA sequence being selected from:

(a) the DNA sequence of any of SEQ ID Nos 12 to 16 or the complementary strands thereof;

(b) DNA sequences which selectively hybridize the DNA sequences defined in (a) or fragments thereof; and

(c) DNA sequences which, but for the degeneracy of the genetic code, would hybridize to the DNA sequences defined in (a) or (b) and which sequences code for a protein or peptide having the same amino acid sequence;

an expression vector comprising a DNA sequence of the invention operably linked to a regulatory sequence;

a host cell transformed with a DNA sequence of the invention or an expression vector of the invention;

a process for producing a protein or peptide of the invention, comprising culturing a host cell of the invention under conditions to provide for expression of the desired protein or peptide.

an antibody capable of binding a peptide or protein of the invention and a method of treating an individual by immunotherapy using the antibody.

### DESCRIPTION OF THE FIGURES

FIG. 1. The binding of radiolabeled α₂M to 10⁹ bacteria of different strains of S. pyogenes grown to early stationary phase is presented in A (bars represent +SEM, n=3). In B the binding of radiolabeled α₂M to 2×10⁸ KTL3 bacteria was competed with α₂M and with protein G (+/−SD, n=3 ). In C the scatchard plot for the reaction between α₂M and 10⁹ KTL3 bacteria is shown. The shape of the plot suggests two binding sites with different affinities (Kₐ=2.0×10⁸M⁻¹ and 5.3×10⁶M⁻¹ respectively).

FIG. 2. A schematic comparison between protein GRAB and protein G is shown in A. The complete nucleotide and amino acid sequence of grab/protein GRAB is shown in B.

FIG. 3. Different strains of S. pyogenes were subjected to PCR and the results are set out in (A). From all strains, except from the AP9 strain, a product of between 500 and 850 bp in size could be amplified (A). Schematic comparison of the mature protein GRAB (amino acids 34-188 in FIG. 2B) encoded by these strains is shown in B.

FIG. 4. MBP-GRAB was used to inhibit the binding of radiolabeled α₂M to 2×10⁸ KTL3 bacteria. Similarly one synthetic peptide (aa 34-56 in FIG. 2B) was able to compete for the binding of α₂M although less efficiency that MBP-GRAB, while an overlapping peptide (aa 51-68 in FIG. 2B) did not compete for the binding. Bars represent +/−SD, n=3.

FIG. 5. An internal fragment of grab, lacking the part of the gene coding for the cell wall attachment, was cloned into the streptococcal suicide plasmid pFW13 to generate FW-grab. pFW-grab was transformed into KTL3 bacteria, to generate MR4. MR4 was completely devoid of α₂M binding as shown (+SD, n=3).

FIG. 6. The binding of the radiolabeled fibrinogen was measured after trypsin treatment of KTL3 or MR4 bacteria. Some bacteria were preincubated with α₂M (+α₂M) and some were not. As can be seen, preincubation of KTL3 with α₂M protected the M protein, and thus fibrinogen binding, from trypsin degradation. α₂M pretreatment of MR4 did not affect the fibrinogen binding (+SD n=3).

FIG. 7. Radiolabeled and activated SCP was added to KTL3 (1), MR4 (3), or the same bacteria preincubated with α₂M (2 and 4 respectively). The binding of SCP was significantly higher to KTL3 bacteria that had been preincubated with α₂M (+SD, n=3).

FIG. 8. Shows the results of an assay of sheep anti-DSP 18. peptide sera on a GRAB coated plate.

FIG. 9. Shows the results of ELISA using

FIG. 10. Shows the serum antibody response in mice immunised with a protein or peptide of the invention.

FIG. 11. Shows the results of opsonization of log phase group A streptococcus by sera to a protein or peptide of the invention.

### DETAILED DESCRIPTION OF THE INVENTION

The invention relates generally to proteins which bind α₂M. Binding of α₂M to bacteria or proteins can be determined using radiolabeled α₂M. For example, bacteria can be incubated with radiolabeled α₂M. After centrifugation, radioactivity of the pellets can be determined and expressed as a percentage of added activity over control samples containing no bacteria. The binding of radiolabeled α₂M could also be competed with non-labeled α₂M or other protein such as protein G. This suggests that the novel protein binds to the same site as does protein G or an overlapping site on α₂M. It suggests that the α₂M binding of Group A streptococcus (GAS) bacteria is attributable to a protein G-like protein. This is confirmed by the examples below, which suggest that protein GRAB is the only α₂M binding protein of GAS.

The Examples below also describe the generation of a mutant strain of Group A Streptococcus, S. pyogenes which no longer expresses protein GRAB on its surface. This could also be used as a control. Binding of α₂M to proteins can be assessed by immobilizing the proteins on a support such as nitrocellulose and probing with radiolabeled α₂M. After washing, the radioactivity of the bound protein can be determined to give an indication of specific binding of α₂M to bound protein. The Examples below describe one method for evaluation of the binding of α₂M to both bacteria or proteins.

The inventors have identified a region of protein GRAB which can inhibit α₂M binding to S. pyogenes which express protein GRAB. The sequence for this region is set out in SEQ ID No. 1. The invention relates to proteins comprising the amino acid sequence of SEQ ID No.1 and variants of this sequence. The term variants is used to cover related amino acid sequences which may differ from the exact sequence of SEQ ID No. 1. Variants according to the invention can be identified in a number of different ways as explained in more detail below.

In another aspect of the invention, a protein or peptide is provided to generate an immune response, preferably a protective immune response to group A streptococcus in an individual. Preferably, the group A streptococcus (S. Pyogenes) is one which expresses protein GRAB as defined herein. A protein or peptide for use in a vaccine formulation is one which is capable of generating an immune response in an individual. Suitable proteins or peptides are derived from protein GRAB or variants thereof. Such proteins or peptides for use in a vaccine formulation may or may not retain the ability to bind α₂M. A protein or peptide of the invention may also be used to generate an antibody to protein GRAB which may be used in the diagnosis or treatment by immunotherapy of GAS infection.

Variant sequences may be identified in protein GRAB produced from other strains of S. pyogenes. Partial sequence data for protein GRAB isolated from a number of different strains of S. pyogenes is set out in SEQ ID Nos. 7-11. Each of these sequences includes the sequence of SEQ ID No. 1 except for a single residue difference in protein GRAB derived from AP1 (SEQ ID No 9). The variation from SEQ ID No. 1 is the replacement of isoleucine for threonine at position 18. This sequence is one example of a variant sequence of the invention.

The Examples below show expression of protein GRAB from a number of other strains of S. pyogenes. Protein GRAB from these strains may also be used to identify an α₂M binding region or a region which inhibits α₂M binding to protein GRAB expressing S. pyogenes, and also to identify sequences which are variants of SEQ ID No. 1. The relevant region from such protein GRABs can be identified by alignment of the amino acid sequence data obtained for protein GRAB from other strains with the sequences set out in SEQ ID Nos 1 - 1. When the maximum alignment is achieved, the relevant region of the protein comprising a variant on SEQ ID No. 1 can readily be identified.

In an alternative aspect of the invention, proteins and variant sequences are those which can be used in a vaccine formulation and against which an immune response, preferably a protective immune response to group A streptococcus is generated on administration of the peptide to an individual. In this aspect of the invention, the protein or peptide may no longer retain the ability to bind α₂M. Such sequences maybe derived as described below to identify sequences which do bind α₂M but may be modified such that the ability to bind α₂M is lost through deletion, substitution or insertion in the amino acid sequence of a protein which does maintain the ability to bind α₂M. Particularly preferred are fragments of protein GRAB which are described in more detail below.

Protein GRAB from other S. pyogenes strains can be identified firstly by investigating the α₂M binding properties of the strain. Subsequently the desired sequence information can be obtained by cloning the genomic DNA and conducting PCR using primers which hybridize to sections of DNA encoding the peptides set out in SEQ ID Nos 1-11. The Examples below demonstrate identification and partial sequencing of protein GRAB derived from a number of S. pyogenes strains. In particular, primers hybridizing to the sequences set out in SEQ ID Nos. 17-21 can be used in the cloning and sequencing of protein GRAB from other S. pyogenes strains. The region of protein GRAB identified in SEQ ID No. 1 is highly conserved between the different strains of S. pyogenes. In general the variant sequences derived from other S. pyogenes would be expected to differ by 1, 2, 3, 4, or up to 5 amino acids from SEQ ID No 1, and more likely by 1 or 2 amino acid residues. Proteins having this variant sequence retain the ability to bind to α₂M.

Variants of SEQ ID No. 1 also include sequences which vary from SEQ ID No.1 but which are not necessarily derived from naturally occurring protein GRAB. These variants may be described as having a % homology to SEQ ID No. 1 or having a number of substitutions within this sequence. Alternatively a variant may be encoded by a polynucleotides which hybridizes to any one of SEQ ID No 12-16, which is discussed in more detail below.

A variant of SEQ ID No. 1 is one which has at least 78% homology thereto. Preferably the variant will be at least 83 or 87% and more preferably 91 or 96% homologous thereto. Methods of measuring protein homology are well known in the art and it will be well understood by those of skill in the art that in the present context, homology is calculated on the basis of amino acid identity (“hard homology”).

Amino acid substitutions may be made, for example from 1, 2 or 3 up to 4, 5 or 6 substitutions in SEQ ID No. 1. The modified sequence generally retains the ability to bind α₂M. Conservative substitutions may be made, for example according to the following Table:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Amino acids in the same block in the second column and preferably in the same line in the third column may be substituted for each other.

Preferably, the proteins of the invention comprise an extension to SEQ ID No. 1. Thus the protein preferably comprises SEQ ID No.2. The protein may also comprise sequences which are fragments of SEQ ID No.2 which incorporate at least all of SEQ ID No 1. The protein may therefore comprise a sequence of 25 amino acids commencing at the N-terminal of SEQ ID No.2 and may comprise 30, 35, 40, 45 or 50 amino acids of SEQ ID No. 2 up to the entire sequence of 58 amino acids of SEQ ID No 2. The proteins of the invention may also comprise variants of such sequences.

The variants can be defined in a similar manner to the variants if SEQ ID No. 1. Thus the variants may comprise variant sequences derived from other strains of S. pyogenes. For example the Examples describe protein GRAB derived from a number of different strains of S. pyogenes. SEQ ID Nos. 7-11 set out sequence data for some of these strains. Alignment with SEQ ID No.2 to give the maximum identity in alignment will allow those of skill in the art to determine variant sequences of SEQ ID No. 2.

Other variants can be identified as outlined above from other S. pyogenes strains by looking for α₂M binding and cloning and sequencing as before. α₂M binding of variant proteins can be determined by expression cloning and western blotting of the recombinant protein using radiolabeled α₂M.

Variants can also be identified by % homology or have substitutions as described above. A greater number of substitutions or lower % homology can be tolerated for longer sequences such as larger fragments of SEQ ID No. 2 or the entire sequence. For example, 1, 2, 3 up to about 10 to 15 substitutions in SEQ ID No.2 may be incorporated. Alternatively a variant may have at least 74%, 78% or 81% homology, and preferably has at least 85% or 90%, 95%, 97% or 98% homology. As before the variants preferably maintain the ability to bind α₂M.

The proteins of the invention may also comprise the sequence of SEQ ID No 3 or a variant sequence thereof, or a fragment of either sequence. Preferably the proteins of the present invention further comprise two or more tandem repeats of the sequence SEQ ID No.3 and variants thereof. The proteins isolated from S. pyogenes and termed protein GRAB have at least two repeated sequences adjacent to the C-terminus of SEQ ID No.2 or variant thereof. These repeat sequences have the sequence set out in SEQ ID No.3 or a variant thereof. As can be seen from SEQ ID Nos 7-11, the sequence can show some variation within each repeat both in a single protein GRAB and also between protein GRAB isolated from different strains of S. pyogenes. Thus the term repeat as used herein does not mean that an exact repeat of the same sequence is present but simply that a sequence and one or more variants thereof are present, preferably in tandem.

The protein may comprise 2, 3, 4, 5 or 6 or more repeat sequences. Each repeat sequence is generally 28 amino acids in length but may be from 21 up to 35 amino acids in length. Within each protein the length of the repeat sequence therein may vary. For example a protein may comprise a sequence of 28 amino acids followed by a variant repeat sequence of 35 amino acids. The repeat sequence of the invention may adapt a coiled coil structure. This structure is based on heptadic structure of amino acid units which allow the protein to form a coil.

Variants of the repeat sequence of SEQ ID No 3 derived from other strains of S. pyogenes can be readily identified by reference to the sequences set out in SEQ ID Nos. 7-11. Each of these sequences has at least two repeats. Repeat sequences derived from protein GRAB from other S. pyogenes strains can be identified as outlined above through cloning and sequencing. Other variants encompassed by the present invention are sequences identified by % homology or substitutions as outlined above for SEQ ID No.1 or Seq ID No. 2. For example a variant may be a repeat having at least 60% homology, preferably at least 70 or 75% up to 85 or 90% up to at least 96% homology with SEQ ID No 3. A variant may have 1, 2 or 3 up to 6, 7, 8 or 9 substitutions in SEQ ID No 3. Preferably the variant retains the heptad structure allowing the region to form a coiled structure. A sequence encoded by a polynucleotide which hybridizes with a polynucleotide encoding a repeat sequence as described herein is also a variant of the invention.

The proteins of the invention may also comprise additional regions such as a cell membrane anchor region and a transmembrane region. The sequence of SEQ ID No.4 comprises a protein having an α₂M binding region, a repeat sequence region and a cell membrane anchor region and transmembrane region. The proteins of the invention can comprise variants of the cell membrane anchor and transmembrane regions as defined above for the other sequences of the protein. Such variants preferably retain the cell membrane anchor function and/or transmembrane function.

It may be desirable to ensure that the transmembrane regions or anchor regions are not present in the protein. For example, if a protein is desired which has the ability to bind α₂M but which will he excreted from the bacterial cell in which it is expressed, the anchor and transmembrane regions are preferably not expressed as part of the protein.

In one preferred embodiment of the present invention, the protein consists essentially of any one of SEQ ID Nos 1-11 and variants thereof as defined above.

The present invention also relates to peptides comprising a fragment of at least 6 amino acids in length of a protein of the invention. In particular, the invention relates to such a peptide comprising a fragment of the protein having the sequence of any one of SEQ ID Nos. 1-11 and variants thereof. Preferably, the fragment will be at least 10, for example at least 12 or 15, amino acids in length. The fragment may be up to 20, 30, 40, 60 or 150 amino acids in length.

In a preferred embodiment, a peptide of the invention has the ability to bind α₂M. This binding can be determined as outlined above. As will be readily appreciated by one skilled in the art, peptides of shorter length preferably comprise a fragment of protein GRAB derived from S. pyogenes. For longer peptides, the sequences may show greater variation as set out above, such as a smaller % homology or greater number of substitutions.

In an alternative aspect of the invention, a peptide has the ability to generate an immune response on administration to an individual and preferably to generate a protective immune response in an individual. Such a peptide may additionally retain the ability to bind α₂M. However, such binding is not necessarily required. A peptide for use in this embodiment comprises a fragment of the protein having the sequence of any one of SEQ ID Nos. 1-11 and variants thereof as described above. Such a fragment is at least 6 amino acids in length and preferably the fragment will be at least 10, for example at least 12 or 15 up to 20, 30 or 40 amino acids in length. Longer fragments such as fragments up to 60 or 150 amino acids in length may also be used. A variant of the sequences of the SEQ ID Nos. 1-11 are described above with reference to the ability to bind α₂M. However such variants for use in a vaccine composition do not need to retain the ability to bind α₂M. Such a variant sequence for use in a vaccine is one which has the ability to generate an immune response on administration to an individual.

Preferably, a peptide for incorporation into a vaccine formulation is one which is derived from the extra cellular region of protein GRAB. Preferred peptides include DSP18, SEQ ID No. 22; EKL 24, SEQ ID No. 23; EKL18, SEQ ID No 24; EER17, SEQ ID No 25 and KKT18, SEQ ID No. 26. Preferred peptides also include variants of these peptides and fragments of the proteins of the invention which incorporate part or all of SEQ ID 22 to 26. In a particular preferred embodiment, the invention relates to a peptide which is derived from the region of protein GRAB located C-terminal and adjacent to the α₂M binding region. Such a peptide is exemplified by the peptide of SEQ ID No. 23, 24 or 25. In one aspect of the invention, a peptide for use in a vaccine composition does not retain the ability to bind α₂M. Binding to α₂M site may reduce the effectiveness of the peptide if there is a large amount of free α₂M which may simply bind to such administered peptide and reduce its efficacy as a vaccine composition, or bind to GRAB in vivo obscuring the target epitope.

Peptides for use in a vaccine composition in accordance with the invention may comprise longer peptide sequences derived from protein GRAB or may encompass the full length protein. Preferably however the vaccine composition comprises a fragment of protein GRAB as defined above. A peptide for use in generating an immune response may be identified by immunisation studies. For example a candidate peptide may be administered to an animal and subsequently the antibody or T-cell response generated which is specific for the peptide may be determined. Antiserum generated following administration of a peptide to an animal may be evaluated for the ability to bind the peptide or to bind protein GRAB. Subsequently the animal may be challenged with Group A streptococcus to evaluate whether a protective immune response has been generated.

In another embodiment, the peptide comprises a fragment of the repeat sequence or variant thereof, as described above. In this embodiment the peptide may comprise an entire repeat sequence that is of about 28 amino acids in length as outlined above, or two or more repeat sequences in tandem.

Proteins and polypeptides of the invention may be in substantially isolated form. It will be well understood that the proteins or peptides may be mixed with carriers or diluents which will not interfere with the intended purpose of the protein or peptide and still be regarded as substantially isolated. A protein or peptide of the invention may also be in substantially purified form, in which case it will generally comprise the protein or peptide in a preparation in which more than 90%, for example more than 95%, 98% or 99%, by weight of the protein or peptide in the preparation is a protein or peptide of the invention.

Proteins or peptides of the invention may be modified for example by the addition of one or more histidine residues to assist in their identification or purification or by the addition of a signal sequence to promote their secretion from a cell. Some of the signal sequences derived from protein GRAB from a number of S. pyogenes strains are set out in SEQ ID Nos. 7-11, and can be seen located N-terminally from the α₂M binding region or SEQ ID No. 1 or variant thereof. It may be desirable to provide the peptides or proteins in a form suitable for attachment to a solid support. The proteins or peptides may thus be modified to enhance their binding to a solid support for example by the addition of a cystine residue.

A protein or peptide of the invention may be labelled with a revealing label. The revealing label may be any suitable label which allows the protein or peptide to be detected. Suitable labels include radioisotopes such as ¹²⁵I, ³⁵S or enzymes, antibodies, polynucleotides and linkers such as biotin. Labelled proteins and peptides of the invention may be used in assays for example to assess levels of α₂M. In such assays it may be preferred to provide the peptides attached to a solid support. The present invention also relates to such labelled and/or immobilized protein and peptides packaged in the form of a kit in a container. The kit may optionally contain other suitable reagent(s), control(s) or instructions and the like.

The proteins of the present invention may be isolated from S. pyogenes expressing the protein. Proteins and peptides of the invention may be prepared as fragments of such isolated proteins. The proteins and peptides of the invention may also be made synthetically or by recombinant means. The amino acid sequence of proteins and polypeptides of the invention may be modified to include non-naturally occurring amino acids or to increase the stability of the compound. When the proteins or peptides are produced by synthetic means, such amino acids may be introduced during production. The proteins or peptides may also be modified following either synthetic or recombinant production.

The proteins or peptides of the invention may also be produced using D-amino acids. In such cases the amino acids will be linked in reverse sequence in the C to N orientation. This is conventional in the art for producing such proteins or peptides.

A number of side chain modifications are known in the art and may be made to the side chains of the proteins or peptides of the present invention. Such modifications include, for example, modifications of amino acids by reductive alkylation by reaction with an aldehyde followed by reduction with NaBH, amidination with methylacetimidate or acylation with acetic anhydride.

The invention also relates to polynucleotides encoding the proteins and peptides of the invention and their use in producing the proteins and peptides of the invention by recombinant means. In particular the invention relates to (a) the DNA sequence of any one of SEQ ID Nos 12 to 16 or the complementary strands thereof (b) DNA sequences which hybridize to the DNA sequences defined in (a) or fragments thereof; and (c) DNA sequences which, but for the degeneracy of the genetic code, would hybridize to the DNA sequences defined in (a) or (b) and which sequences code for a polypeptide having the same amino acid sequence. Hybridization is typically carried out under conditions of high stringency, such as hybridization buffer of 6×SSC, 0.5% SDS at 65° C. Hybridization conditions equivalent to the conditions described herein could also be used to identify the polynucleotides of the invention.

Polynucleotides of the invention may also comprise corresponding RNA to these DNA sequences. The polynucleotides may be single or double stranded. They may also be polynucleotides which include within them synthetic or modified nucleotides. A number of different types of modification to oligonucleotides are known in the art. These include methylphosphonate and phosphorothioate backbones, addition of acridine or polylysine at the 3′ and/or 5′ ends of the molecule. For the purposes of the present invention, it is to be understood that the polynucleotides described herein may be modified by any method available in the art.

Preferred polynucleotides of the invention include polynucleotides encoding any of the proteins and peptides described above. Those skilled in the art will understand that numerous different polynucleotides can encode the same protein or peptide as a result of degeneracy of the genetic code.

A nucleotide sequence capable of selectively hybridizing to the DNA sequence of any one of SEQ ID Nos: 12 to 16 or to a DNA sequence complementary to any one of those sequences will be generally at least 70%, preferably at least 80 or 90% and more preferably at least 95% or 97%, homologous to such a DNA sequence. This homology may typically be over a region of at least 20, preferably at least 30, for instance at least 40, 60 or 100 or more contiguous nucleotides of the said DNA sequence.

Any combination of the above mentioned degrees of homology and minimum sized may be used to define polynucleotides of the invention, with the more stringent combinations (i.e. higher homology over longer lengths) being preferred. Thus for example a polynucleotide which is at least 80% homologous over 25, preferably over 30 nucleotides forms one aspect of the invention, as does a polynucleotide which is at least 90% homologous over 40 nucleotides.

Homologues of polynucleotide or protein sequences are referred to herein. Such homologues typically have at least 70% homology, preferably at least 80, 90%. 95%, 97% or 99% homology, for example over a region of at least 15, 20, 30, 100 more contiguous nucleotides or amino acids. The homology may calculated on the basis of amino acid identity (sometimes referred to as “hard homology”).

For example the UWGCG Package provides the BESTFIT program which can be used to calculate homology (for example used on its default settings).

(Devereux et al (1984) Nucleic Acids Research 12. p387-395). The PILEUP and BLAST algorithms can be used to calculate homology or line up sequences (such as identifying equivalent or corresponding sequences (typically on their default settings), for example as described in Altschul S. F. (1993) J Mol Evol 36:290-300; Altschul, S, F et al (1990) J Mol Biol 215:403-10.

Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSP's containing them. The word hits are extended in both directions along each sequence for as far as the cumulative alignment score can be increased. Extensions for the word hits in each direction are halted when: the cumulative alignment score falls off by the quantity X from its maximum achieved value: the cumulative score goes to zero or below, due to the accumulation of one or more negative-scoring residue alignments; or the end of either sequence is reached. The BLAST algorithm paramters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787 One measure of similarity provided by the BLAST algorithm is the smallest sum probability (P(N)), which provides an idication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

Polynucleotides of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein. Examples of primers of the invention are set out in SEQ ID Nos 17 to 21.

Longer polynucleotides will generally be produced using recombinant means, for example using PCR (polymerase chain reaction) cloning techniques. This will involve making a pair of primers (e.g. of about 15-30 nucleotides) to a region of the grab which it is desired to clone, bringing the primers into contact with DNA obtained from a bacterial cell, preferably of an S. pyogenes strain, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Although in general the techniques mentioned herein are well known in the art, reference may be made in particular to Sambrook et al, 1989.

Polynucleotides or primers of the invention may carry a revealing label. Suitable labels include radioisotopes such as ³²P or ³⁵S enzyme labels, or other protein labels such as biotin. Such labels may be added to polynucleotides or primers of the invention and may be detected using techniques known per se.

Polynucleotides or primers of the invention or fragments thereof labelled or unlabelled man be used by a person skilled in the art in nucleic acid-based tests for detecting or sequencing grab in a bacterial sample.

Such tests for detecting generally comprise bringing a bacterial sample containing DNA into contact with a probe comprising a polynucleotide or primer of the invention under hybridizing conditions an detecting any duplex formed between the probe and nucleic acid in the sample. Such detection may be achieved using techniques such as PCR or by immobilizing the probe on a solid support, removing nucleic acid in the sample which is not hybridized to the probe, and then detecting nucleic acid which was hybridized to the probe. Alternatively, the sample nucleic acid may be immobilized on a solid support, and the amount of probe bound to such a support can be detected.

The probes of the invention may conveniently be packaged in the form of a test kit in a suitable container. In such kits the probe may be bound to a solid support where the assay format for which the kit is designed requires such binding. The kit may also contain suitable reagents for treating the sample to be probed, hybridizing the probe to nucleic acid in the sample, control reagents, instructions, and the like.

Polynucleotides of the invention can be incorporated into a recombinant replicable vector. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about the replication of the vector. The vector may be recovered from the host cell. Suitable host cells include bacteria such E. coli, yeast, mammalian cell lines and other eucaryotic cell lines, for example insect cells such as Sf9 cells.

Preferably, a polynucleotide of the invention in a vector is operably linked to a regulatory sequence that is capable of providing for the expression of the coding sequence by the host cell, i.e. the vector is an expression vector. The term “operable linked” refers to a juxtaposition wherein the components described are in a relationship permitting them to function in their intended manner. A regulatory sequence “operably linked” to a coding sequence is ligated in such a way that expression of ale coding sequence is achieved under condition compatible with the control sequences.

Such vectors may be transformed or transfected into a suitable host cell as described above to provide for expression of a polypeptide of the invention. This process may comprise culturing a host cell transformed with an expression vector as described above under conditions to provide for expression by the vector of a coding sequence encoding the polypeptides, and optionally recovering the expressed polypeptides.

The vectors may be for example, plasmid or virus vectors provided with an origin or replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell.

Promoters/enhancers and other expression regulation signals may be selected to be compatible with the host cell for which the expression vector is designed. For example prokaryotic promoters may be used, in particular those suitable for use in E. coli strains. When expression of the polypeptides of the invention is carried out in mammalian cells, mammalian promoters may be used. Tissues-specific promoters, for example hepatocyte cell-specific promoters, may also be used. Viral promoters may also be used, for example the Moloney murine leukaemia virus long terminal repeat (MML V LTR), the promoter rous sarcoma virus (RSV) LTR promoter, the SV40 promoter, the human cytomegalovirus (CMV) IE promoter, herpes simplex virus promoters or adenovirus promoters. All these promoters are readily available in the art.

Vaccines may be prepared from one or more of the proteins or peptides of the invention and a physiologically acceptable carrier or diluent. Typically, such vaccines are prepared as injectables, either as liquid solutions or suspensions; solid forms suitable for solution in, or suspension in, liquid prior to injection may also be prepared. The preparation may also be emulsified, or the protein encapsulated in a liposome. The active immunogenic ingredient may be mixed with an excipient which is pharmaceutically acceptable and compatible with the active ingredient. Suitable excipients are, for example, water, saline, dextrose, gycerol, ethanol, of the like and combinations thereof.

In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminium hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-( 1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against an immunogenic polypeptide containing a GRAB antigenic sequence resulting from administration of this polypeptide in vaccines which are also comprised of the various adjuvants.

The vaccines are conventionally administered parentally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the vaccine composition is lyophilised, the lyophilised material may be reconstituted prior to administration, e.g. a a suspension. Reconstitution is preferably effected in buffer.

Capsules, tablets and pills for oral administration to a patient may be provided with an enteric coating comprising, for example Eudragit “S”, Eudragit “L”, cellulose acetate, cellulose acetate phthalate or hydroxypropylmethyl cellulose.

The proteins or peptides of the invention may be formulated into the vaccine as neutral or salt forms. Pharmaceutically acceptable salts include the acid addition salt (formed with free amino groups of the peptide) and which are formed with inorganic acids such as, for example, hydrochloric or phosphoric acids, or such organic acids such as acetic, oxalic, tartaric and maleic. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine and procaine.

The vaccines are administered in a manner compatible with the dosage formulation and in such amount will be prophylactically and/or therapeutically effective. The quantity to be administered, which is generally in the range of 5 μg to 100 mg, preferably 250 μg to 10 mg of antigen per dose, depends on the subject to be treated, capacity of the subject's immune system to synthesize antibodies and the degree of protection desired. Precise amounts of active ingredient required to be administered may depend on the judgement of the practitioner and may be peculiar to each subject.

The vaccine may be given in a single dose schedule, or preferably in a multiple dose schedule. A multiple does schedule is one in which a primary course of vaccination may be 1-10 separate doses, followed by other doses given at subsequent time intervals required to maintain and or reinforce the immune response for example at 1 to 4 months for a second dose, and if needed, a subsequent dose(s) after several months. The dosage regimen will also, at least in part, be determined by the need of the individual and be dependent upon the judgement of the practitioner.

The proteins and peptides of the invention which have the ability to bind α₂M may be used to purify α₂M from a sample. Typically, the proteins or peptides of the invention will be bound to a solid support. A sample potentially containing α₂M can be applied to the support to remove α₂M from the sample. If desired, α₂M can then be released from the support for further use.

The proteins and peptides of the invention which arc capable of inhibiting binding of α₂M to the surface of streptococci may be used to inhibit such α₂M binding to the bacterial surface. The proteins and peptides can also be used in competition studies to identify other agents which may effect α₂M binding.

The proteins and peptides of the invention can be used to generate antibodies against strains of S. pyogenes. The poylnucleotides of the invention can be used in the production of the proteins and peptides of the invention. As outlined above, they may also be used as primers or probes for identification of related genes to grab.

The nucleotide sequences of the invention and expression vectors can also be used as vaccine formulations as outlined above. The vaccines may comprise naked nucleotide sequences or be in combination with cationic lipids, polymers or targeting systems. The vaccines may be delivered by any technique suitable for delivery of nucleic acid vaccines.

The immunogenic polypeptides prepared as described above can be used to produce antibodies, both polyclonal and monoclonal. If polyclonal antibodies are desired, a selected mammal (e.g., mouse, rabbit, goat, horse, etc.) is immunised with an immunogenic polypeptide of the invention. Serum from the immunised animal is collected and treated according to known procedures. If serum containing polyclonal antibodies to the polypeptide contains antibodies to other antigens, the polyclonal antibodies can be purified by immunoaffinity chromatography. Techniques for producing and processing polyclonal antisera are known in the art.

Monoclonal antibodies directed against Streptococcal epitopes in the polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against polypeptides of the invention can be screened for various properties; i.e., for isotype and epitope affinity. Preferably the antibody is specific for a GRAB protein epitope.

Antibodies, both monoclonal and polyclonal, which are directed against polypeptides of the invention are particularly useful in diagnosis and those which are neutralising are useful in passive immunotherapy. Monoclonal antibodies, in particular, may be used to raise anti-idiotype antibodies. Anti-idiotype antibodies are immunoglobulins which carry an “internal image” of the antigen of the infectious agent against which protection is desired.

Techniques for raising anti-idiotype antibodies are known in the art. These anti-idiotype antibodies may also be useful for treatment of Streptococci, as well as for an elucidation of the immunogenic regions of polypeptides of the invention.

It is also possible to use fragments of the antibodies described above, for example, Fab fragments. Antibodies generated to a peptide of the invention may be administered to an individual to treat GAS infection by passive immuno therapy. The antibodies of the invention may be formulated with a pharmaceutically acceptable carrier and delivered in the same way as set out above for the vaccine compositions. Preferably the antibody is administered in an amount effective to ameliorate GAS infection in the individual.

### EXAMPLES

The following Examples illustrate the invention.

### Example 1

S. pyogenes bind native α2M via a protein G like protein—Different strains of S. pyogenes were tested for their ability to bind radiolabeled native α2M. S. pyogenes strains denoted AP are from the Institute of Hygiene and Epidemiology Prague, Czech Republic. The KTL strains are from the Finnish Institute for health, and the SF370 strain is the ATCC 700294 strain. Bacteria were harvested in early stationary phase or after overnight culture, washed in PBS with 0.05% Tween-20 and 0.02% azide (PBSAT) and resuspended in the same buffer. Concentration of bacteria was determined by spectrophotometry and 2×10⁹ or 4×10⁸ were incubated with radiolabeled α₂M in 225 μl PBSAT for 50 minutes. For competition different amounts of unlabeled inhibitor was added to the tubes. After centrifugation radioactivity of the pellets was determined and expressed as percentages of the added activity deducing the non-specific bindings to the polypropylene tubes.

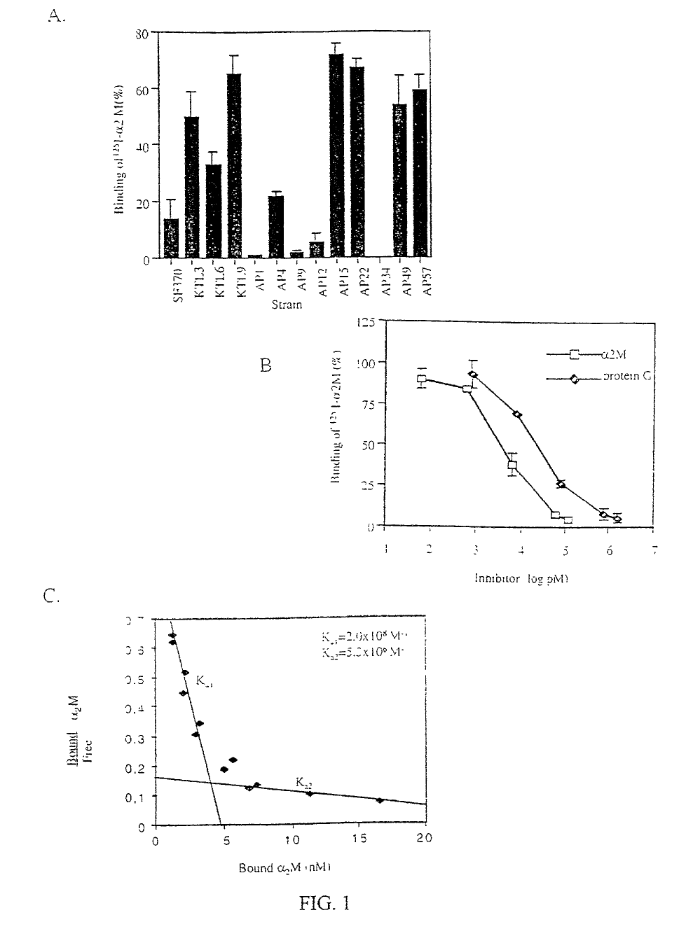

The results are shown in FIG. 1A. The binding ranged from 0-76% and differed between strains even within a given serotype. No strain bound a trypsin complexed form of α₂M (data not shown).

The KTL3 strain of the clinically important M1 serotype which bound 53% of added α₂M was chosen for further studies. The binding of radiolabeled α₂M to the KTL3 strain could be competed by both non-radioactive α₂M and by protein G from the strain G148. a group G Streptococcus (FIG. 1B). The scatchard plot for the reaction between α₂M and KTL3 bacteria (FIG. 1C) suggests that two different affinities exist, one high affinity interaction Kₐ=2.0×10⁸M⁻¹ and one low affinity interaction Kₐ=5.3×10⁶M⁻¹. Since the binding of α₂M to the KTL3 strain could be competed by protein G, we used the protein sequence of protein G from G148 in a tBLASTn search against the Streptococcal Genome Sequencing Project database.

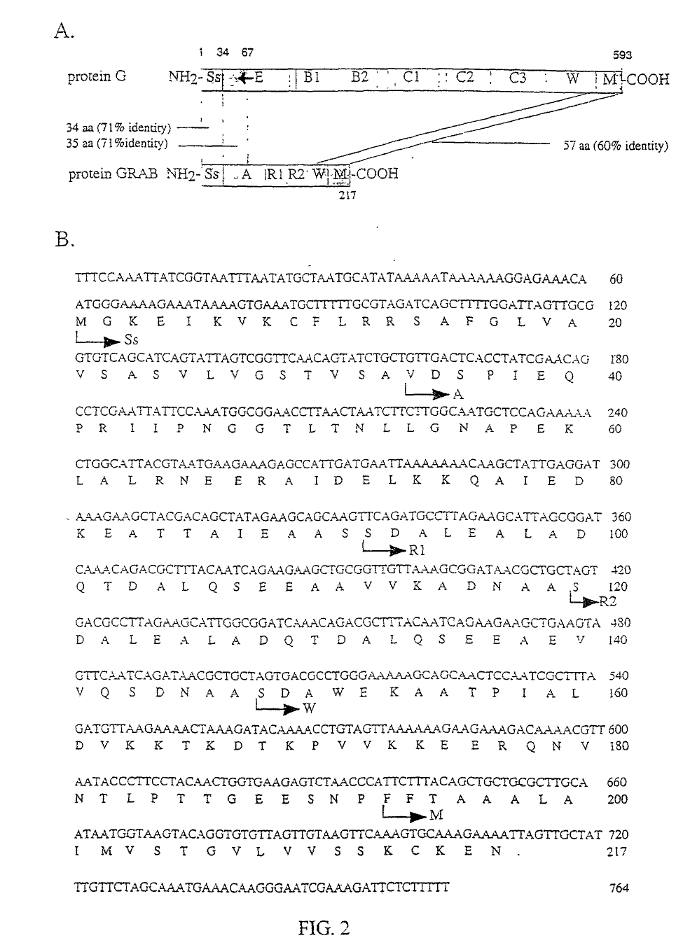

A gene coding for a protein with some homology to the α₂M binding E domain of protein G, as well as to the signal sequence and cell-wall attachment of protein G, was identified. The protein was termed protein GRAB from protein G related α₂M binding protein and consisted of 217 amino acids with a deduced molecular weight of 22.8 kDa. In 2A a schematic representation of the homology between protein GRAB and protein G is shown. In FIG. 2B the nucleotide and amino acid sequences are set out. The A region includes the α₂M binding region. Two repeat regions are identified R1 and R2 and are followed by the wall spanning (W) and membrane spanning (M) regions. Protein GRAB was found to contain the consensus sequence for gram-positive surface cell wall anchored proteins (LPXTGX) followed by a sketch of 19 hydrophobic amino acids and a seven residue long hydrophilic C-terminus (FIG. 2B). The first 34 amino acids of protein GRAB showed some homology to the signal sequence (Ss) of protein & and was followed by 35 amino acids with some homology to the E domain of protein G (FIG. 2B). Spacing the regions with homology to protein G two unique repeated regions of 28 amino acids were identified.

### Example 2

Distribution of expression of grab—Genomic DNA was prepared from S. pyogenes. PCR was performed using Taq polymerase (Gibco-BRL, Gaithersburg, Md.) and synthetic oligonucleotides hybridizing to grab. Primers hybridized to the following nucleotides in FIG. 2B primer 1: 101-125, primer 2: 101-128, primer 3: 160-185. primer 4: 594-563 and primer 5: 627-605. Restriction enzymes and ligase were from Gibco-BRL and standard ligation, transformation, and plasmid isolation methods were used. For PCR screening and for cloning in pGEM (Promega, Madison, Wis.) primers 1 and 5 were used. Sequencing of the pGEM-grab plasmids was performed using an ABI-470 prism and Taq dyed dideoxy terminator kit (Perkin and Elmer, Norwalk, Conn.).

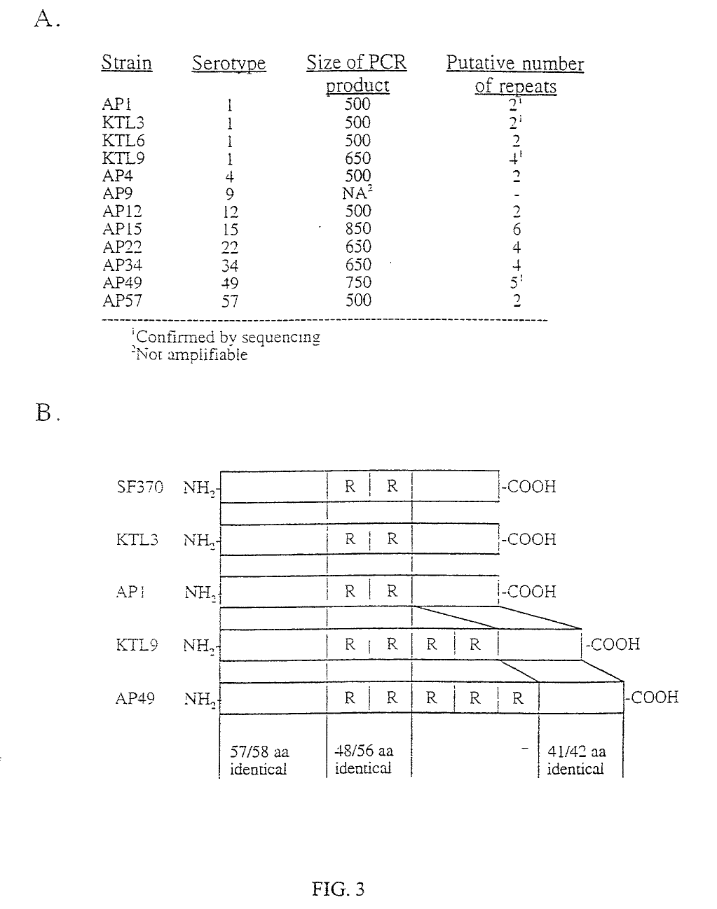

The same strains that were used in the screening for α₂M binding were subjected to PCR using primers hybridizing to grab. A PCR product could be generated from all strains except for the AP9 strain, but the size of the product varied between 500 base pairs (bp) and 850 bp (FIG. 3A). Sequencing of the PCR product from four strains revealed that the size polymorphism was due to a variable number of 28 amino acids repeats (FIG. 3B). Comparing the sequence from these four strains and the one presented in the Streptococcal Genome Sequencing Project it was found that protein GRAB is highly conserved. Both the C- and -terminus was nearly 100% conserved while the repeated region showed 86% identity between strains (FIG. 3B). SEQ ID Nos 7 to 11 show partial sequence data for these strains. SEQ ID Nos 12 to 16 show corresponding nucleotide sequences.

The transcription of grab was investigated using Northern blotting where total RNA from the KTL3 strain which bound radiolabeled α₂M and a strain that did not (AP1) was isolated from bacteria in early logarithmic phase, late logarithmic phase, early stationary phase and late stationary phase. The RNA was electrophorized, blotted, and probed with a PCR product generated from grab using primers 1 and 5. Detectable amounts of a transcript of approximately 600 bp of grab RNA was found in KTL3 bacteria but not in AP1. The expression was highest in early logarithmic phase and dropped to undetectable amounts in the late stationary phase. The same filters were probed with a probe hybridizing with 16S which verified that the same amount of RNA had been applied to each well.

### Example 3

Protein GRAB binds α₂M via the extreme N-terminus—The DNA encoding the predicted mature protein GRAB amino acids 34-189 in FIG. 2B) from the KTL3strain was PCR cloned into the pMal-p2 vector using the EcoRl and Pstl sites present in primers 3 and 5 respectively. The vector was transformed into E. coli. For molecular cloning, purposes the DH5α strain of Escherichia coli was used. E. coli were grown un Luria Bertoni broth (10 g tryptone (Difco), 10 g NaCI, and 5 g yeast extract (Difco)/l) supplemented with 2 g glucose/l when using the pMal-p2 vector. For growth in petri dishes 15 g/l of bacto agar (Difco) was added. When E. coli contained plasmid 100 μg/ml ampicillin (Sigma, St. Louis, Mo.) was added to the medium. A fusion protein between a maltose binding protein (MBP) and protein GRAB was produced upon induction with IPTG.

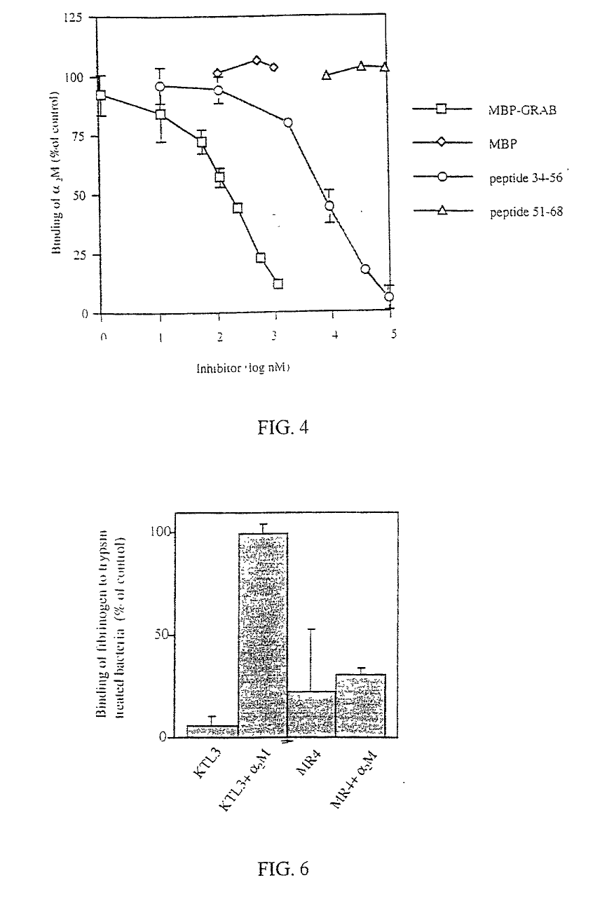

The fusion protein was purified by affinity chromatography on an amylase resin. The fusion, MPB-Grab, Protein G and MSP-α chain of β galactosidase were subjected to SDS-PAGE and stained with commassie. An identical SDS-PAGE was blotted to a nitrocellulose filter, and the filter was probed with radiolabeled α₂M. The predicted size of MBP-GRAB is 60 kD but it migrates with an apparent size of 80 kDa. Both Protein G and the MBP-GRAB fusion were found to bind α₂M while MBP was unable to bind α₂M. Similarly MBP-GRAB, protein G, and MBP were applied in slots to a nitrocellulose membrane and probed with α₂M and it could be concluded that MBP-GRAB bound α₂M while MBP did not. MBP-GRAB, but not MBP, was found to compete for the binding of radiolabeled α₂M to KTL3 bacteria (FIG. 4). Thus both protein GRAB and protein G can inhibit the binding of α₂M to KTL3 bacteria indicating that the two proteins interact with the same epitope in α₂M. A peptide covering the extreme N-terminus of the mature protein GRAB (amino acids 34-56 FIG. 2B SEQ ID No 1) was synthesized and was able to compete for the binding of α₂M to KTL3 bacteria while an overlapping peptide (amino acids 49-68 in FIG. 2B) did not affect the binding (FIG. 4). Thus we conclude that the extreme N-terminus of protein GRAB is responsible for binding of α₂M.

### Example 4

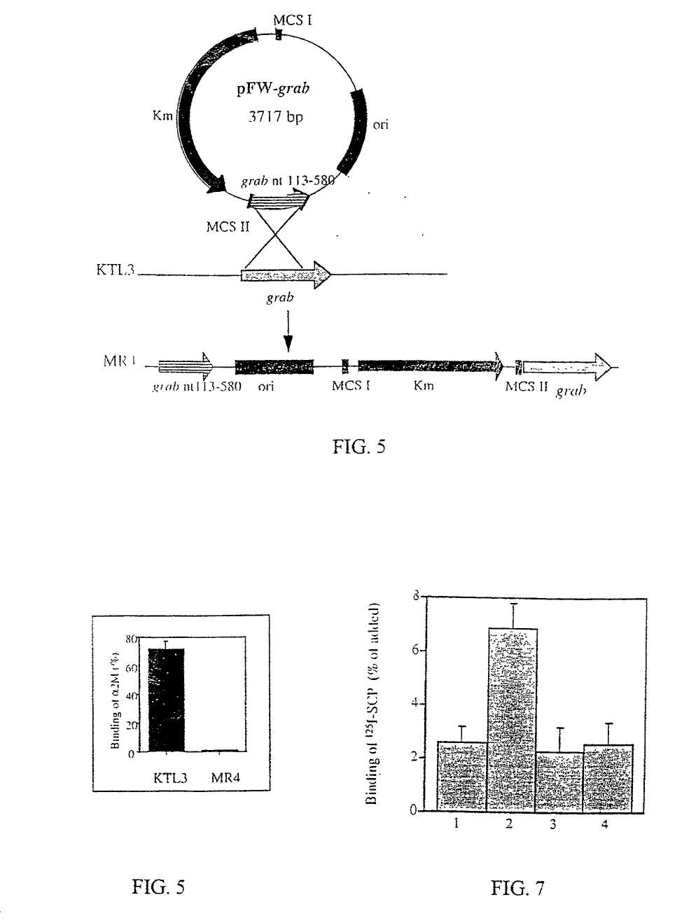

Generation of a mutant devoid of protein GRAB on its surface—A fragment of grab lacking the part encoding the putative cell wall attachment region was generated by PCR from the KTL3 strain using primers 2 and 4. The fragment was cut with Xhol and HindIII which exclusively cut within primers 3 and 4 respectively and cloned into the corresponding site of streptococcal suicide plasmid pFW13 to generate FW-grab. This generated a 468 bp internal fragment (nt 113-580 in FIG. 2B) of grab lacking the part encoding the cell wall attachment (FIG. 5). The plasmid was electroporated into E. coli, plasmid purified and 2 μg of pFW-grab was electroporated into KTL3 bacteria for homologous recombination (FIG. 5) and several kanamycin resistant transformants were obtained. Using this cloning strategy the mutant should be devoid of surface bound protein GRAB and instead secrete a truncated form (amino acids 34-174 in FIG. 2B). One transformant called MR4 was selected and its ability to bind radiolabeled α₂M was completely abolished (FIG. 5).

When the supernatants from an overnight culture of MR4 and KTL3 were precipitated with TCA, subjected to SDS-PAGE, blotted to nitrocellulose, and probed with radiolabeled α₂M it was found that the MR4 strain secreted an α₂M binding protein of 32 kDa which was not found in the KTL3 medium. The predicted size of the mature protein GRAB is 14.9 kDa, but apparently it migrates much slower in SDS-PAGE which is in concordance with the observation that the MBP-GRAB fusion also migrates slower than predicted. MR4 and KTL3 bacteria had similar growth characteristics in THY medium and the mutant survived as well as the wild type in fresh human blood (data not shown).

### Example 5

Hybridization protocol is carried out as follow. Streptococci were grown in Todd-Hewitt broth with 0.2% yeast extract (THY) in 5% CO₂ at 37° C. Genomic DNA was prepared from S. pyogenes. 20 μg of DNA was cleaved by EcoRI and subjected to agarose gel electrophoresis and capillary blotting onto Hybond-N filters (Amersham, Amersham, UK). A probe was generated by PCR using Taq polymerase and synthetic oligonucleotides with sequences GACTCACCTATCGAACAGCCTCG and AGCTTCTTCTGATTGTAAGCG, hybridising to grab. The PCR product was purified on a MicroSpin™ S-200 HR column and was radio labeled with [α-32P]dATP using bacteriophage T4 polymerase. Membrane was prehybridized in a solution of 6×SSC. 0.5% SDS, 5×Denharts solution, and 100 μg/ml salmon sperm DNA at 50° C. for two hours. Probe was boiled for five minutes and added to a solution of 6×SSC, 0.5% SDS and 5×Denharts solution and incubated for 14 hours at 65 ° C. This was followed by washing at room temperature in 2×SSC+0.5% SDS for five minutes and 2×SSC+0.1% SDS for 15 minutes. Further washes were performed in 0.1×SSC+0.5% SDS at 37° C. for one hour and in 0.1×SSC+0.1% SDS at 53° C. for 30 minutes. Filter was air dried followed by exposure on BAS-III imaging plate and scanning with Bio-Imaging Analyser BAS-2000.

### Example 6

α₂M is active and protects the M protein from tryptic digestion when bound to protein GRAB—10⁹ KTL3 or MR4 cells were incubated for 40 minutes with 20 μg α₂M and carefully washed with PBS. Bound α₂M was eluted using 0.1 glycine pH 2 and subjected to SDS-PAGE. In parallel, 0.3 μg of trypsin was added to the α₂M treated bacteria and the trypsin was allowed to react with surface bound α₂M for 5 minutes. Free trypsin (not in complex with α₂M) was blocked by adding a fourfold molar excess of SBTI. Cells were pelleted by centrifugation and the resulting pellet was washed once in 1 ml of PBS and resuspended in 150 μl PBS supplemented with 40 μg of chloramphenicol/ml. The remaining activity of trypsin in the supernatant and the resuspended pellet was determined using the chromogenic substrate Nα-bensoyl-L-arginine p-nitroanilide (L-BAPNA) at a concentration of 0.25 mg/ml by measuring OD₄₀₅ after three hours. The obtained value for MR4 was subtracted from that of KTL3 and compared to a standard, where the same assay was run in parallel using purified α₂M of known concentration (0.5 μg). For protection assays bacteria were preincubated with α₂M as above, treated with 0.1 μg of trypsin in PBS with chloramphenicol as above for 60 minutes at 37° C. Bacteria were diluted 10 times in PBSAT supplemented with 10 mM benzamidine and chloramphenicol as above and 2×10⁶ bacteria were subjected to a binding assay using radiolabeled fibrinogen.

It was found that roughly 0.5 μg of α₂M was bound to 10⁹ KTL3 bacteria while no band was seen in the eluate from MR4. In parallel, the amount of active α₂M bound was estimated by calculating the amounts of α₂M trapped trypsin. This L-BAPNA assay showed-that 10⁹ KTL3 bacteria bound 0.27+/−0.03 μg of α₂M, which correlates well with what could be eluted from the bacteria.

The complex between trypsin and α₂M was released from the KTL3 surface since all trypsin activity was found in the supernatant. To determine if this was due to release of th trypsin-α₂M complex from protein GRAB or tryptic degradation of protein GRAB, KTL3 cells were treated with trypsin and SBTI, washed, incubated with α₂M, and bound α₂M was eluted. No α₂M was bound to the trypsin treated cells indicating that protein GRAB was digested by trypsin. Thus it was concluded that α₂M bound to the surface of KTL3 is active and that protein GRAB is sensitive to trypsin treatment.

A characteristic of S. pyogenes M-proteins are their susceptibility to trypsin degradation. This led us to investigate whether preincubation of KTL3 bacteria with α₂M could protect the M protein, and thus fibrinogen binding, from proteolytic degradation by trypsin. It was found that the fibrinogen binding of KTL3 could be preserved by α₂M pretreatment, while the fibrinogen binding of MR4 was unaffected by α₂M pretreatment (FIG. 6).

### Example 7

SCP is tripped by α₂M in solution or α₂M bound to S. pyogenes—Radiolabeled SCP was activated in activation buffer (1 mM EDTA, and 10 mM DTT in 0.1 M NaAc-HAc, pH 5.0) for 30 minutes at 40° C. Activated SCP (4 μl) was mixed with either 4 μg α₂M or 2 μl of plasma in 20 μl PBS, allowed to react for 15 minutes at 37° C., and subjected to SDS-PAGE using non-reducing conditions followed by autoradiography. Alternatively 2×10⁹ bacteria were pretreated with 40 μg α₂M, washed, and incubated with radiolabeled and activated SCP for 15 minutes. Bacteria were pelleted by centrifugation and pellet was washed with 2 ml of PBSAT and recentrifuged. Radioactivity of the pellet was measured and bound material was released by suspension of pellet in non-reducing SDS-PAGE sample buffer. Eluate was subjected to SDS-PAGE and autoradiography.

As outlined above, radiolabeled and activated SCP was mixed with either purified α₂M or with plasma and subjected to non-reducing SDS-PAGE and autoradiography. Radiolabeled SCP and α₂M were separated on the same gel as a reference. Part of the radioactivity could be seen as a band with the apparent size of α₂M indicating that a covalent complex had been formed between SCP and α₂M. Pretreatment of KTL3 and MR4 with α₂M resulted in an increased binding of SCP to KTL3, but not MR4, bacteria (FIG. 7). When bound material was eluted from these bacteria, subjected to SDS-PAGE and autoradiography (as before using radiolabeled SCP and α₂M as a reference), it was found that SCP was in complex with α₂M in the case of KTL3. but not in MR4. The supernatants were separated on the same gel, and a small proportion of the radioactivity from the α₂M pretreated KTL3 bacteria, could be seen as band with the apparent size of α₂M (data not shown). Thus we conclude that α₂M in solution or bound to S. pyogenes via protein GRAB can trap, and probably also inhibit SCP.

### Example 8

Generation of protein GRAB antiserum. The part of protein GRAB encoding aa 34-188 (FIG. 2B) was PCR amplified from the KTL3 strain and cloned into the pET11d vector (Pharmacia Biotech, Uppsala, Sweden). Sequencing of the plasmid insert confirmed that the cloned gene was identical to grab from the KTL3 strain. Resulting Escherichia coli (BL21, Pharmacia Biotech) transformants were grown in 2×YT to OD₆₂₀ of 0.5 and induced using 0.5 mM IPTG. Bacteria were harvested after 3 hours by centrifugation and resuspended in 20 mM Tris-HCl pH 8. Bacteria were sonicated and recentrifuged at 8000×g. The bacterial lysate was subjected to ion-exchange chromatography using a mono Q column and a FPLC system (Pharmacia Biotech). Protein GRAB could be purified to approximately 90% homogeneity.

100 μg of protein GRAB, from the ion exchange chromatography, in 500 μl saline was supplemented with 330 μl complete and 170 μl incomplete Freund's adjuvans and material was used to immunize one rabbit. Rabbit was boostered after 6 weeks with 100 μg of protein GRAB in 500 μl saline supplemented 500 μl incomplete Freund's adjuvans. Blood was drawn 2 weeks after boostering and serum was prepared. Serum was used in ELISA experiments where 1 ng of protein GRAB or malose bindings protein (MBP, purified from the same strain of E. coli) in 50 mM carbonate buffer, pH 9.6 was absorbed to Maxisorb plates (Nunc) at 4° C. overnight. Wells were blocked for 1 hour at room temperature using 200 μl of PBS+0.05% Tween 20 (PBST), 1% (w/v) BSA (Sigma) and incubated with varying amounts of protein GRAB antiserum or preimmune serum in the same buffer for 2 hours. This was followed by five rounds of washing with PBST and incubation with a peroxidase labelled goat antirabbit antibody (1:3000 in PBST+1% BSA) for 1 hour at room temperature. After another round of washing 200 μl of developing solution (1 mg ABTS and 6 mg hydrogen peroxide/ml of Na-citrate pH 4.5) was added to each well and OD₄₀₅ was determined after 20 minutes of incubation at room temperature. Values over 0.3 were regarded as positives. Titer of the preimmune serum was <1:100 and titer of the immune serum was >1:128 000 for protein GRAB and 1:4000 for MBP.

Similarly KTL3 or MR4 bacteria were heat killed at 65° C. and 10⁸ bacteria were absorbed (as above) to each well. ELISA was performed as above with the exception that protein A (1:5000) was used instead of the secondary antibody. Titer of the preimmune serum was 1:200 for KTL3 and 1:100 for MR4. Titer of the immune serum was 1:4000 for KTL3 and <1:1000 for MR4,

The antiserum was further used for western blotting of a membrane prepared as in Example 4. The filter was blocked for 30 minutes at 37° C. using PBST with 5% skimmed milk. Immune or preimmune serum was diluted 1:1000 in the blocking buffer and the filter was incubated for 30 minutes at 37° C. The filter was subsequently washed three tines for 10 minutes at 37° C. using PBST with 0.5M NaCl. Incubation with a peroxidise labelled goat anti rabbit antibody (1:3000 in blocking buffer) was performed for 30 minutes at 37° C. followed by washing as above. Membranes were incubated with freshly made substrate consisting of 500 μl of 44.4 mM p-Coumaric acid. 100 μl 250 mM Luminol (5-amino-2-3-dihydro-1,4-phtalazinedione), and 6.1 μl of 30% H₂O₂ dissolved in 20 ml Tris-HCl pH 8. Membranes were incubated for one minuted at room temperature, dried and put in a plastic bag for exposure on XAR film (Kodak). The preimmune serum showed no reactivity, whereas the immune serum specifically reacted with a band of the same size as the α₂M-binding protein in Example 4.

### Example 9

The purpose of this study was to determine whether sheep immunised with various GRAB peptides, produced IgG antibodies which have the ability to bind to the corresponding peptide or native GRAB protein and whether the IgG antibodies could be titrated out. 1 mg of the relevant peptide in 1.3 ml saline and 3.25 ml of Freund's complete/incomplete adjuvant was used for each immunisation.

Boosters were given at 3 weeks intervals with 0.5 mg peptide in 1.3 ml saline and 3.25 ml adjuvant. The immunisation mixture was injected at 6 subcutaneous sites for each sheep. The peptides used were as follows:

| | |
|---|---|
| Spy-PG-EKL24 (37-61) | EKLALRNEER AIDELKKQAI EDKE |
| | C*-COOH |
| Spy-PG-EKL 18 (37-55) | EKLALRNEER AIDELKKQ C* -COOH |
| Spy-PG-EER 17 (44-61) | EERAIDELKK QAIEDKE C* -CCOH |
| Spy-PG-DSP 18 (13-31) | DSPIEQPRII PNGGTLTN C* -COOH |
| Spy-PG-KKT 19 (141-160) | KKTKDTKPVV KKEERQNVN C* -COOH |

C* cysteine insert for attachment to a hetero-bifunctional linker. Peptides are linked to KLH.keyhole limpet hemocyanin.

Titration and Inhibition ELISA Protocol for Analysis of Anti-GRAB Peptide Anti-Sera

GRAB protein was coated onto microtiter plates (100 μl/well) at a concentration of 1 μg/ml, in 0.05M carbonate-bicarbonate buffer pH 9.6. The plates were incubated for 1 hour at 37° C. The plates were then washed ×5 with PBS-T (250 μl/well) and blocked with 1% BSA/PBS-T (100 μl/well) for 1 hour at 37° C.

After washing the plates ×5 with PBS-T, pre and post immune sera from sheep immunised with peptide conjugate vaccine candidates including FCA/Spy-PG-EKL24-KLH, FCA/Spy-PG-EKL 18-KLH, FCA/Spy-PG-EER 17-KLH. FCA/Spy-PG-DSP18-KLH and FCA/Spy-PG-KKT19-KLH were diluted from 1/100 to 1/1,000,000 in PBS-T. The sera were then incubated on the GRAB coated plates (100 μl sera/well) for 1 hour, at 37° C. The plates were washed ×5 with PBS-T and incubated with donkey anti-sheep IgG/peroxidase conjugate (1/1000 in PBS-T) for 1 hour at 37° C. The plates were then incubated with 0.1 mg/ml TMB substrate (100 μl/well) for 10 minutes and then the reaction was stopped with 2M H₂SO₄ (50 μl/well). Absorbances were read at 450 nm.

For an inhibition ELISA, post immune sera from sheep immunised with the peptide conjugates mentioned above, were pre-incubated at 37° C., for 1 hour, at a dilution of 1/10,000, with the corresponding free peptide at concentrations ranging from 0 to 10 μg/ml. For controls, post immune sera (1/10,000) were incubated with Spy-PH-QKQ19 (10 μg/ml). This peptide has the sequence QKQQQLETEKQISEASRKS C* —COOH. The serum peptide mixtures were then assayed on GRAB coated plates, as previously indicated.

In a following inhibition ELISA post immune sheep sera were incubated with the corresponding raw peptide or the Spy-PH-QKQ19 control peptide, at a concentration of 100 μg/ml.

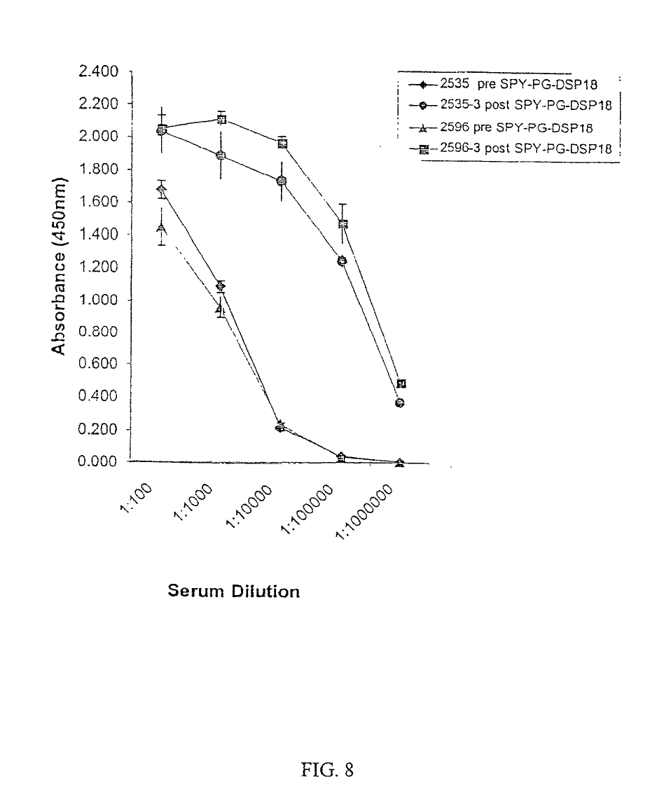

At dilutions of 1/100 and 1/1000, absorbances of both pre and post immune sera were generally similar. Large differences between pre and post immune serum absorbances, were often observed at dilutions of 1/10,000 and 1/100,000. FIG. 8 shows the results of the assay of sheep anti-DSP18-peptide sera on a GRAB coated plate.

Inhibition ELISA's confirmed that in all cases GRAB binding antibodies in the post immune sera, could be prevented from binding whole GRAB protein, by the addition of the corresponding raw peptide. The results of the ELISA where 100μ/ml of raw peptide was added to the sera is shown in Table 1 below. When 100μ/ml of raw peptide was added to the sera. % inhibition levels generally exceeded 80%, demonstrating that high proportions of the IgG antibodies present in the serum samples were peptide specific.

TABLE 1
Inhibition of anti GRAB peptide antibody binding
to GRAB protein, by raw peptide (100 μg/ml)

| | | Mean absorbance 450 nm | | | % Inhibition | |
|---|---|---|---|---|---|---|
| | | | 100 μg/ml | 100 μg/ml | 100 μg/ml | 100 μg/ml |
| | Peptide | | relevant | control | relevant | control |
| Sheep No. | Immunogen | Unabsorbed | peptide | peptide | peptide | peptide |
| 2529-3 | Spy-PG-EKL24 | 1.719 | 0.321 | 1.694 | 81.3 | 1.5 |
| 2530-3 | Spy-PG-EKL24 | 1.924 | 0.274 | 1.730 | 85.8 | 10.1 |
| 2531-3 | Spy-PG-EKL18 | 1.515 | 0.207 | 1.598 | 86.3 | 0 |
| 2532-3 | Spy-PG-EKL18 | 1.508 | 0.267 | 1.538 | 82.3 | 0 |
| 2533-3 | Spy-PG-EER17 | 1.693 | 0.297 | 1.454 | 82.5 | 14.1 |
| 2631-3 | Spy-PG-EER17 | 1.656 | 0.259 | 1.590 | 84.4 | 4.0 |
| 2535-3 | Spy-PG-DSP18 | 1.591 | 0.201 | 1.665 | 87.4 | |
| 2596-3 | Spy-PG-DSP18 | 1.752 | 0.492 | 1.676 | 71.9 | 4.3 |
| 2597-3 | Spy-PG-KKT19 | 0.763 | 0.177 | 0.780 | 76.8 | 0 |
| 2598-3 | Spy-PG-KKT19 | 1.469 | 0.210 | 1.689 | 85.7 | 0 |

NB. The control peptide used was Spy-PH-QKQ19

Titration ELISA Protocol

Peptide was coated onto microtiter plates (100 μl/well) at a concentration of 5 μg/ml, in 0.05M carbonate-bicarbonate buffer pH 9.6. The plates were incubated for 1 hour at 37° C. The plates were then washed ×5 with PBS-T (250 μl/well) and blocked with 1% BSA/PBS-T (100 μl/well) for 1 hour at 37° C.

After washing the plates ×3 with PBS-T, pre and post immune sera from sheep immunised with peptide conjugate vaccine candidates including FCA/Spy-PG-EKL24-KLH, FCA/Spy-PG-EKL18-KLH, FCA/Spy-PG-EER 17-KLH, FCA/Spy-PG-DSP 18-KLH and FCA/Spy-PG-KKT19-KLH were diluted from 1/100 to 1/1,000,000 in PBS-T. The sera were then incubated on plates coated with the corresponding peptide (100 μl sera/well) for 1 hour, at 37° C. The plates were washed ×3 with PBS-T and incubated with donkey anti-sheep IgG/peroxide conjugate (1/1000 in PBS-T) for 1 hour at 37° C. After washing ×5 with PBS-T, the plates were incubated with 0.1 mg/ml TMB substrate (100 μl/well) for 10 minutes and then the reaction was stopped with 2M H₂SO₄ (50 μl/well). Absorbances were read at 450 nm.

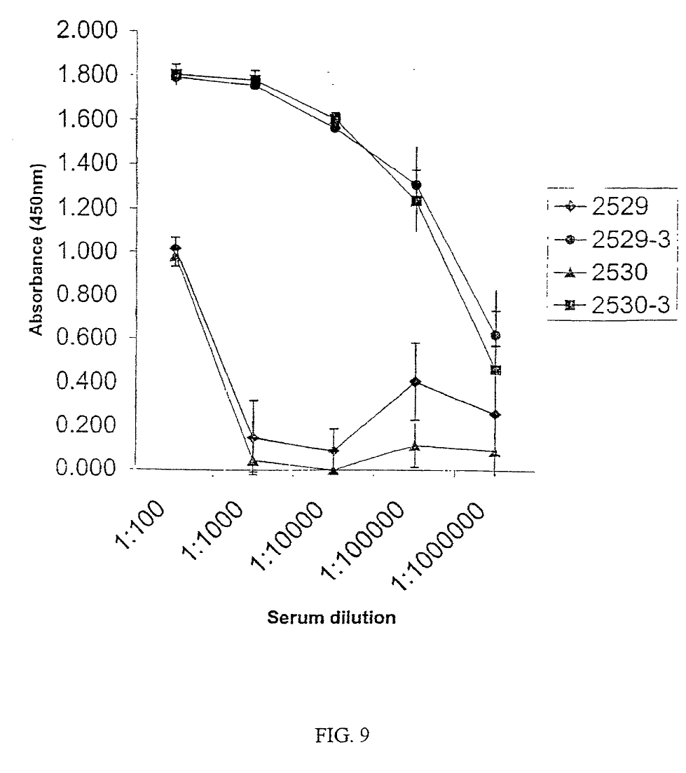

The results obtained from the experiment described above for Spy-PG-EKL 24 are presented in FIG. 9 and have been summarised for all peptides in table 2 below.

TABLE 2
Sheep anti-GRAB peptide antibody titres as determined by ELISA

| | | | Assay | |
|---|---|---|---|---|
| Sheep | | | asborsbance | |
| No. | Pre/post | Peptide immunogen | cut off point | Antibody Titre |
| 2529 | pre | | 0.45 | 1 in 100 |
| 2529-3 | post | Spy-PG-EKL24 | 0.45 | 1 in 1,000,000 |
| 2530 | pre | | 0.45 | 1 in 100 |
| 2530-3 | post | Spy-PG-EKL24 | 0.45 | 1 in 1,000,000 |
| 2531 | pre | | 0.45 | 1 in 100 |
| 2531-3 | post | Spy-PG-EKL18 | 0.45 | 1 in 100 |
| 2532 | pre | | 0.45 | 1 in 1000 |
| 2532-3 | post | Spy-PG-EKL18 | 0.45 | 1 in 1,000,000 |
| 2533 | pre | | 0.45 | 1 in 1000 |
| 2533-3 | post | Spy-PG-EER17 | 0.45 | 1 in 1,000,000 |
| 2631 | pre | | 0.45 | 1 in 100 |
| 2631-3 | post | Spy-PG-EER17 | 0.45 | I in 100,000 |
| 2535 | pre | | 0.30 | 1 in 1000 |
| 2535-3 | post | Spy-PG-DSP18 | 0.30 | 1 in 1,000,000 |
| 2596 | pre | | 0.30 | 1 in 1000 |
| 2596-3 | post | Spy-PG-DSP18 | 0.30 | 1 in 1,000,000 |
| 2597 | pre | | 0.15 | 1 in 1,000,000 |
| 2597-3 | post | Spy-PG-KKT19 | 0.15 | 1 in 1,000,000 |
| 2598 | pre | | 0.15 | 1 in 1000 |
| 2598-3 | post | Spy-PG-KKT19 | 0.15 | 1 in 1,000,000 |

### Example 10

Immunisation Studies

Mice (5-6 weeks old), 10 per group, were immunised subcutaneously (sc) at the tail base with 50 μl of vaccine emulsion containing 30 μg of peptide/protein/peptide-conjugates emulsified in CFA. Control mice are given PBS in CFA. Peptides emulsified in Complete Freund's adjuvant (CFA, H37Ra, Difco Laboratories Cat# 3113-60-5) were prepared as follows:

33 μl of peptide (10 mg/ml stock) and 517 μl of sterile PBS with 550 μl of CFA were mixed in an eppendorf. Using a 1 ml syringe with 18G needle materials was homogenized until the volume was reduced by half. Mixture may be tested by centrifuging in eppendorf for 1 min at 1000 rpm, if mixture does not separate it is OK to proceed. Alternatively, one drop of emulsion is placed on water. If emulsified, drop should remain tight and not disperse. The emulsified mixture was drawn into same syringe, tuberculin needle fixed, and air bubbles removed.

Mice were given booster injections at days 23 and 30, so, of 30 μg and 15 μg respectively of peptide/protein/peptide conjugates dissolved in PBS.

Mice were bled at Day 14, Day 23, Day 29 and Day 38 via the tail artery and sera are prepared and stored at −20° as follows. Murine blood was collected into eppendorf tubes (100-300 μl) via scalpel cut to the tail artery. The blood was allowed to clot either overnight at 4° C. or for 1 hour at 37° C. The blood clot was picked out and discarded with sterile toothpick or pipette tip, eppendorf tube was spun at 3000 rpm for 10 minutes. The sera was removed (clear supernatant) to fresh tube. Short term storage <1 week at 4° C., long term storage at −20° C. ELISA was carried out as described below in Example 11.

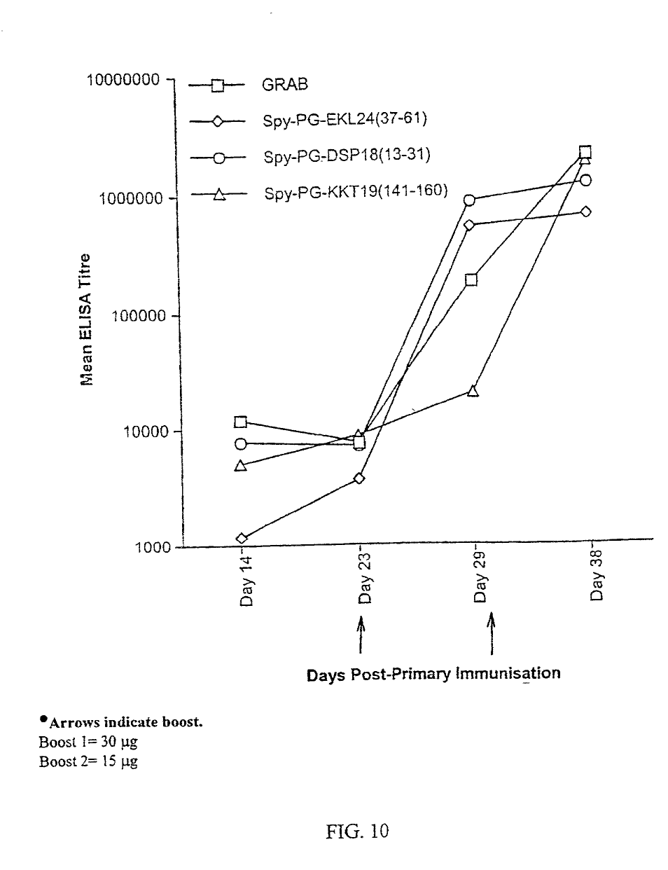

The results are shown in FIG. 10.

Opsonization was carried out using blood from the day 47 bleed. A 100 μl aliquot of stock GAS (Group A streptococcus) was cultured overnight in 5 ml of sterile Todd Hewitt Broth (THB)/1% Neopeptone at 37° C. To use log phase growth bacteria in the assay, 20 μl of the overnight GAS culture was subinoculated into 5 ml of THB/1% Neopeptone that was pre-warmed at 37° C. GAS were grown at 37° C. for 2 hours. GAS (either log phase GAS [2 hour culture] or stationary phase GAS [from the overnight culture]) were diluated in sterile saline to 10⁻⁵.

Fifty μl of the 10⁻⁴ and 10⁻⁵ bacterial dilution was mixed with 50 μl of heat inactivated (60° C. for 10 minutes) normal mouse serum or immune mouse serum, mixed well, and incubated at room temperature for 20 minutes.

400 μl of normal heparinised human blood (pre tested to be non-opsonic for the strain of GAS used in the assay) was added and the mixture was incubated with end-to-end rocking at 37° C. for 3 hours. 50 μl of the bacterial dilution was plated out mixed in a petri-dish with 15 ml molten 2.5% blood THB agar. 50 μl of the 10⁻⁴ bacterial dilution was held at 4° C. until it was plated out to estimate inoculum size. The plates were incubated at 37° C. overnight. Mean colony count was determined by counting colonies on plates. The percentage reduction in colony-forming units (CFU) of bacteria is calculated by comparing means colony counts after incubation with mouse immune serum compared with normal mouse serum multiplied by the dilution factor.

Tests were carried out as follows:

Number of sera tested in the opsonisation assay per immunisation group:

GRAB protein n=3

EKL24-KLH, n=10

DSP18-KLH, n=6

KKT19-KLH, n=10

PepM 88/30 n=3. Sera from mice immunised with PepM derived from 88/30 GAS was used as the positive control in the assay.

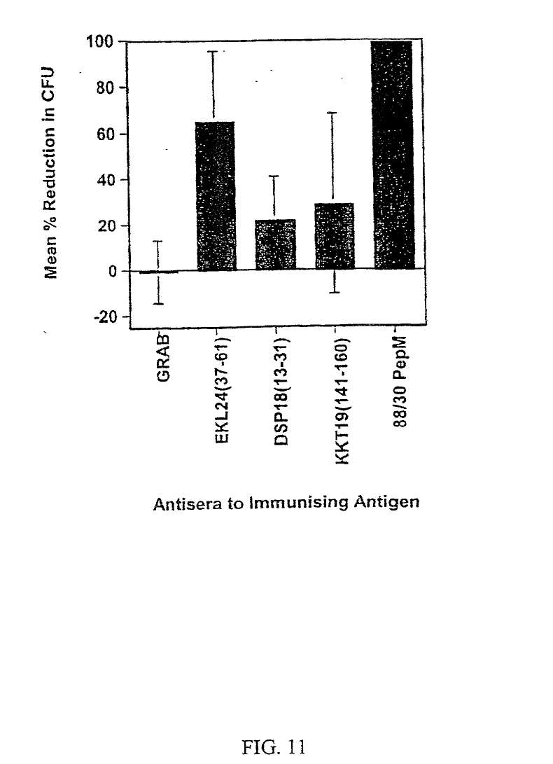

The results are shown in FIG. 11. The mean is ±sem.

### Example 11

Additional studies were carried out specifically to look for an antibody in human sera having specificity to a C-terminally adjacent region to the α₂M binding site of protein GRAB. Such an antibody should be able to bind to protein GRAB as this region should be available on the surface of GAS. In view if the studies described in Example 10. EKL24 was studied further in a human population. Human studies focussed on the pre-existing immunity of an endemic human population (Thailand) highly exposed to group A streptococcal infections. Table 3 below shows that sera from Thai individuals with rhematic heart disease (RHD) and healthy individuals with no heart disease, alike, both contained antibodies to EKL24. Antibody titers were measured by ELISA as set out below.

Antigen (peptide/protein) was diluted to 5 μg/ml in carbonate-bicarbonate buffer. For example, 5 μl of peptide from a 10 mg/ml peptide stock was added to 10 ml of carbonate-biocarbonate buffer (enough for one plate). 100 μl per well was added to flat bottomed polyvinyl chloride microplates (Flow Laboratories Inc.) and incubated overnight at 4° C. or 90 mins at 37° C. Antigen was flicked off the plate and the wells were blocked with 200 μl of 5% skim milk in PBS-Tween 20 overnight at 4° C. or 90 mins at 37° C. Plates were washed 3 times with PBS-Tween 20. Human or mouse sera are diluted 1:100 in the first row and serially diluted 1:2 down the plate to 1:12800 in a final volume of 100 μl.

Plates with primary antibody are then incubated at 37° C. for 90 minutes. Plates are washed 5 times with PBS-Tween 20. If using human sera, goat anti-human IgG/HRP (Bio-rad) or if using mouse sera, goat anti-mouse IgG (Amrad) was diluted 1:3000 in 0.5% Skim milk/PBS-Tween 20. 100 μl is added to each well and incubated at 37° C. for 90 minutes. Plates are washed 5 times with PBS-Tween 20. 100 μl of OPD substrate (OPD FAST, Sigma-OPD and buffer tablets supplied with kit) was added to each well and incubated in the dark at room temperature for 30 minutes. The optical density was measured at 450 nm. For human antibodies, antigen-specific antibody concentration is calculated using standard curves of optical density versus known concentrations of human IgG for murine antibodies, a value of titer is used to measure quantity of antibody and is defined as the mean plus three standard deviations of the normal mouse sera wells.

The results are set out in Table 3 below.

TABLE 3
Serum Antibody Response to peptide Spy-PG-EKL24 (37-61) in Control
and RHD Thais.

| Number of Individuals with an antibody | | |
|---|---|---|
| response: | RHD | Controls |
| Titre ≦ 400 | 45/62 | 19/35 |
| Titre 800-1600 | 8/62 | 16/35 |
| Titre ≧ 3200 | 9/62 | 0/35 |

ELISA was used to measure human serum antibodies to the peptide. The titer is defined as the mean plus three standard deviations of the blank (no antibody) wells.

Subsequently, T-cell proliferation assays were carried out.

Thirty ml of heparinised blood is split between two 50 ml Falcon tubes with conical base and diluted 1:2 (15 ml) in sterile PBS. Blood is underlayed with 10 ml of Ficoll (at room temperature).

Cells are separated by centrifugation at room temperature, 1200 rpm for 30 minutes (with brake off). PBMC layer from both tubes are removed with a sterile pipette and pooled into a single 50 ml Falcon tube, diluted to 50 ml of sterile PBS and centrifuged at 1500 prm for 10 minutes.

The supernatent is discarded and PBMC are resuspended in 5 ml sterile RPMI/10% normal human sera (NHS). NHS has been heat-inactivated for 20 minutes at 60° C. added to media to 10% in RPMI and filter-sterilised prior to use in the assay.

Cells are counted and resuspended in RPMI/10%NHS with 100 μg/ml streptomycin/1000 U/ml penicillin /2.5 mcg/ml fungizone (CSL:catalogue # 0929501), a final concentration of 1×10⁶ cells/ml.

Peptides/proteins were plated out onto round bottomed 96 well plates at predetermined optimal concentrations (30 μg/well of peptide). Wells without antigen were also included 200 μl of PBMC, at a final concentration of 2×10⁵ cell/well, were added to 96 well plates containing the peptide/proteins. After 4 days of culture at 37° C. in 5% CO₂, 25 μl of culture supernatant can be removed from each well for cytokine analysis. After 6 days of culture 0.25 μCi ³H methyl-thymidine was added to each well and 16 hours later incorporation of label was measured by liquid scintillation spectroscopy. Cells are harvested onto a filter mat, filters are dried then sealed in plastic bags with 12 ml of scintillant and counted in a LKB Wallac 1205 Betaplate liquid scintillation counter. The mean cpm of triplicate wells with peptide/protein was divided by the mean cpm of 6 wells without peptide to give a stimulation index (SI). A SI of 5 was used as a cut-off for significant proliferative response in adult subjects as previously described (Pruksakorn S, Currie B. Brandt E, Phornphutkul C. Hunsakunachi S, Manmontri A, Robinson J H, Kehoe M A, Galbraith A. Good M F. Int. Immunol. 1994; 6: 1235-44).

The assay demonstrated that PBMC from RHD patient in the population recognised EKL24. Results are set out in Table 4 below.

TABLE 4
Proliferative response of PMBC From Control and RHD Thais to peptide
Spy-PG-EKL24 (37-61).

| Number of Individuals with a | | |
|---|---|---|
| Stimulation Index >5 | RHD | Controls |
| | 4/62 | 0/35 |

The sequences mentioned herein are set out in the sequence listing below and can be summarised as follows:

SEQ ID No. 1 is the amino acid sequence of positions 34-56 inclusive of strain SF370 as set out in FIG. 2B.

SEQ ID No. 2 is the amino acid sequence of positions 34-91 inclusive of strain SF370 as set out in FIG. 2B.

SEQ ID No. 3 is the amino acid sequence of positions 92-119 inclusive of strain SF370 as set out in FIG. 2B and is one of the repeat sequences of the protein.

SEQ ID No. 4 is the amino acid sequence of positions 34-217 inclusive of strain SF370 as set out in FIG. 2B and is the full length mature protein i.e. without the signal sequence.

SEQ ID No. 5 is the amino acid sequence of positions 34-174 inclusive of strain SF370 as set out in FIG. 2B. This truncated form of the protein is missing the trans-membrane and wall anchor regions.

SEQ ID No. 6 is the amino acid sequence of positions 34-193 inclusive of strain SF370 as set out in FIG. 2B, and does not include the membrane spanning region of the protein.

SEQ ID No. 7 is the amino acid sequence of the full length protein of strain SF370 as set out in FIG. 2B including signal sequence.

SEQ ID Nos. 8-11 are partial amino acid sequences for protein GRAB derived from strains KTL9, AP1, AP49 and KTL3 respectively.

SEQ ID Nos. 12-16 are DNA sequences encoding the amino acid sequences of SEQ ID Nos. 7-11 respectively.

SEQ ID Nos. 17-21 are primers derived from SEQ ID No. 12.

SEQ ID No. 22 is the amino acid sequence for the peptide DSP18

SEQ ID No. 23 is the amino acid sequence for the peptide EKL 24

SEQ ID No. 24 is the amino acid sequence for the peptide EKL 18

SEQ ID No. 25 is the amino acid sequence for the peptide EER 17

SEQ ID No. 26 is the amino acid sequence for the peptide KKT 19

1. A protein which is capable of binding to α₂M and which comprises the amino acid sequence of SEQ ID No 1 or a functional variant thereof.

2. A protein according to claim 1 comprising the amino acid sequence of SEQ ID No 2 or a functional variant thereof.

3. A protein according to claim 1 or claim 2 further comprising one or more tandem repeats having the amino acid sequence of SEQ ID No 3 or a variant thereof.

4. A protein according to any one of claims 1, 2 or 3 further comprising a cell membrane anchor region together with a hydrophobic transmembrane region.

5. A protein according to any preceding claim consisting of the amino acid sequence of any of SEQ ID Nos 1 to 11 or a variant thereof.

6. A peptide comprising a fragment of at least 6 amino acids in length of the protein of claim 5.

7. A peptide according to claim 6 comprising a fragment of at least 20 amino acids of the protein of claim 5.

8. A peptide according to claim 6 or 7 which is capable of generating an immune response against group A streptococcus.

9. A peptide according to claim 6 or 7 which binds α₂M.

10. A peptide according to claim 6 or 7 comprising the acid sequence of SEQ ID NO: 3 or a variant of the said sequence.

11. A peptide according to claim 10 comprising two or more repeats of the amino acid sequence of SEQ ID NO: 3 or of a variant of the said sequence.

12. A protein or peptide which is capable of generating a protective immune response to group A streptococcus which comprises:
(i) the amino acid sequence of SEQ ID No. 1
(ii) a functional of (i)
(iii) a functional fragment of at least 6 amino acids in length of (i) or (ii).

13. A DNA sequence which codes for a protein or peptide according to any preceding claim, said DNA sequence being selected from:
(a) the DNA sequence of any of SEQ ID Nos 12 to 16 or the complementary strands thereof;
(b) DNA sequences which selectively hybridize the DNA sequences defined in (a) or fragments thereof; and
(c) DNA sequences which, but for the degeneracy of the genetic code, would hybridize to the DNA sequences defined in (a) or (b) and which sequences code for a protein or peptide having the same amino acid sequence.

14. An expression vector comprising a DNA sequence according to claim 13 operably linked to a regulatory sequence.

15. A host cell transformed with the DNA sequence of claim 13.

16. A host cell according to claim 15 transformed with the expression vector of claim 14.

17. A process of producing a protein or peptide according to any of claims 1 to 12, comprising culturing a host cell as defined in claim 15 or 16 under conditions to provide for expression of the desired protein or peptide.

18. A vaccine composition comprising a protein or peptide according to claim 12 and a pharmaceutically acceptable carrier.

19. A protein or peptide according to any one of claims 1 to 12 for use in generating a protective immune response in an individual to group A streptococcus.

20. A method of immunising an individual against a group A streptococcus comprising administering a protein or peptide according to claim 12 to the individual.

21. An antibody capable of binding to a peptide or protein according to any one of claims 1 to 12.

22. A method of treating an individual with a GAS infection comprising administering an antibody according to claim 21 to said individual.
